(19) United States
(12) Patent Application Publication
(10) Pub. No.: US 2026/0287602 A1
(43) Pub. Date: Sep. 24, 2026

(54) FLUORESCENT SENSORS FOR MARIJUANA BREATH TEST

(71) Applicants: University of Utah Research Foundation, Salt Lake City, UT (US); Gentex Corporation, Zeeland, MI (US)

(72) Inventors: Ling Zang, Salt Lake City, UT (US); Yanke Che, Sandy Country, UT (US); Jiangfan Shi, Salt Lake City, UT (US)

(21) Appl. No.: 19/573,024

(22) Filed: Mar. 20, 2026

Related U.S. Application Data
(60) Provisional application No. 63/775,101, filed on Mar. 20, 2025.

(51) Int. Cl. G01N 33/94; G01N 21/64

(52) U.S. Cl. CPC G01N 33/948; G01N 21/6428

(57) ABSTRACT

A molecular sensor for detecting marijuana, includes a compound having a structure of Y-[D]ₙ-A-Z, D is an electron donor group according to Formula (I):

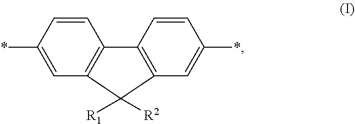

A is an electron acceptor group according to Formula (II):

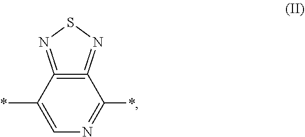

Y is R₃, R₃-A, or R₃-D-A, wherein Z is R₃ or [D]ₘ-R₃, n is an integer between 1 to 5, m is an integer between 1 to 3, R₁ and R₂ are each an alkyl chain, R₃ is an alkoxyphenyl end group.

### CROSS-REFERENCE TO RELATED APPLICATION

This application claims priority to and the benefit under 35 U.S.C. § 119(e) of U.S. Provisional Patent Application No. 63/775,101, filed on Mar. 20, 2025, entitled FLUORESCENT SENSORS FOR MARIJUANA BREATH TEST, the entire disclosure of which is hereby incorporated herein by reference.

### FIELD OF THE DISCLOSURE

The present disclosure generally relates to a sensor and detection method for marijuana, and more particularly to molecular sensors and methods for detecting marijuana.

### BACKGROUND

The primary psychoactive component of marijuana is tetrahydrocannabinol (THC), which stimulates the brain response to pleasure. The other major component present in marijuana is cannabidiol (CBD), which is not psychoactive, rather better recognized for its medical applications. While both THC and CBD can be considered as molecular markers of marijuana, THC has been the dominant target to be monitored due to its physiological activity.

### SUMMARY OF THE DISCLOSURE

According to one aspect of the present disclosure, a molecular sensor for detecting marijuana, includes a compound having a structure of Y-[D]ₙ-A-Z, D is an electron donor group according to Formula (I):

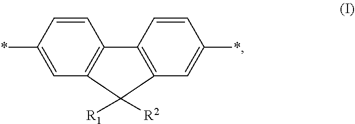

A is an electron acceptor group according to Formula (II):

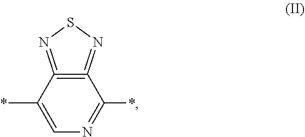

Y is R₃, R₃-A, or R₃-D-A, wherein Z is R₃ or [D]ₘ-R₃, n is an integer between 1 to 5, m is an integer between 1 to 3, R₁ and R₂ are each an alkyl chain, R₃ is an alkoxyphenyl end group.

According to another aspect of the present disclosure, a fluorescent sensor for detecting tetrahydrocannabinol (THC) or cannabidiol (CBD) includes, a porous film, the porous film includes an oligomer including fluorescent electron donor-acceptor molecules in a conjugation structure, a housing, an inlet and an outlet, the porous film is positioned between the inlet and the outlet, a light excitation source, and a fluorescence detector.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a schematic diagram of an example fluorescent sensor for detecting marijuana, according to aspects of the present disclosure;

FIG. 2 is a schematic diagram of another example fluorescent sensor for detecting marijuana, according to aspects of the present disclosure;

FIG. 3 is a flowchart illustrating an example method of detecting a marijuana breath marker, according to aspects of the present disclosure;

FIG. 4 is a reaction scheme showing a synthesis route for synthesizing an example fluorescent sensor compound, 4,7-bis(9,9,9′,9′-tetrahexyl-7′-(4-methoxyphenyl)-9H,9′H-[2,2′-bifluoren]-7-yl)-[1,2,5]thiadiazolo[3,4-c]pyridine, according to aspects of the present disclosure;

FIG. 5 is a graph of a ¹H-nuclear magnetic resonance (¹H-NMR) spectrum of 4,7-bis(9,9,9′,9′-tetrahexyl-7′-(4-methoxyphenyl)-9H,9′H-[2,2′-bifluoren]-7-yl)-[1,2,5]thiadiazolo[3,4-c]pyridine, according to aspects of the present disclosure;

FIG. 6 is a graph of a matrix-assisted laser desorption ionization-time of flight mass spectrometry (MALDI-TOF-MS) spectrum of 4,7-bis(9,9,9′,9′-tetrahexyl-7′-(4-methoxyphenyl)-9H,9′H-[2,2′-bifluoren]-7-yl)-[1,2,5]thiadiazolo[3,4-c]pyridine, according to aspects of the present disclosure;

FIG. 7A is a graph of a fluorescence quenching response to THC vapor at different temperatures dependent on time, according to aspects of the present disclosure;

FIG. 7B is a graph of a fluorescence response to CBD vapor at different temperatures dependent on time, according to aspects of the present disclosure;

FIG. 8A is a fitted curve and equation of the fluorescence quenching response for THC at different vapor pressures, according to aspects of the present disclosure;

FIG. 8B is a fitted curve and equation of the fluorescence quenching response for CBD at different vapor pressures, according to aspects of the present disclosure;

FIG. 9A is a graph of the fluorescence response to acetone, a common potential interferent, according to aspects of the present disclosure;

FIG. 9B is a graph of the fluorescence response to ethanol, a common potential interferent, according to aspects of the present disclosure;

FIG. 9C is a graph of the fluorescence response to isopropanol, a common potential interferent, according to aspects of the present disclosure;

FIG. 9D is a graph of the fluorescence response to water of relative humidity, a common potential interferent, according to aspects of the present disclosure;

FIG. 9E is a graph of the fluorescence response to hexane, a common potential interferent, according to aspects of the present disclosure;

FIG. 9F is a graph of the fluorescence response to heptane, a common potential interferent, according to aspects of the present disclosure;

FIG. 9G is a graph of the fluorescence response to octane, a common potential interferent, according to aspects of the present disclosure;

FIG. 10A is a graph of the fluorescence response of TFT-C6 thin film towards 1 ng, 2 ng, 5 ng, and 10 ng of THC, according to aspects of the present disclosure;

FIG. 10B is a graph of the fluorescence response of TFT-C6 thin film towards 5 ng, 10 ng, and 20 ng of THC, according to aspects of the present disclosure;

FIG. 10C is a graph of the fluorescence response of TFT-C4 thin film towards 1 ng, 2 ng, 5 ng, and 10 ng of THC, according to aspects of the present disclosure;

FIG. 10D is a graph of the fluorescence response of TFT-C4 thin film towards 5 ng, 10 ng, and 20 ng of THC, according to aspects of the present disclosure;

FIG. 11A is a linear fitting curve for the limit of detection (LOD) calculation of the fluorescence response of TFT-C6 towards vaporized THC, according to aspects of the present disclosure;

FIG. 11B is a linear fitting curve for the limit of detection (LOD) calculation of the fluorescence response of TFT-C4 towards vaporized THC, according to aspects of the present disclosure;

FIG. 12A is a graph of the fluorescence response of the compound of Structure XXIII towards 0.01 ng, 0.6 ng, 1 ng, and 1.5 ng of THC, according to aspects of the present disclosure;

FIG. 12B is a linear fitting curve for the limit of detection (LOD) calculation of the fluorescence response of the compound of Structure XXIII towards THC, according to aspects of the present disclosure;

FIG. 13A is a graph of a detector response of 1 μl saliva deposited on a polytetrafluoroethylene (PTFE) testing strip, according to aspects of the present disclosure; and

FIG. 13B is a graph of a detector response of 1 μl saliva co-deposited with varying amounts of THC (10 pg, 20 pg, 100 pg) on a polytetrafluoroethylene (PTFE) testing strip, according to aspects of the present disclosure.

### DETAILED DESCRIPTION

The present illustrated embodiments reside primarily in combinations of method steps and apparatus components related to molecular sensors and methods for detecting marijuana. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

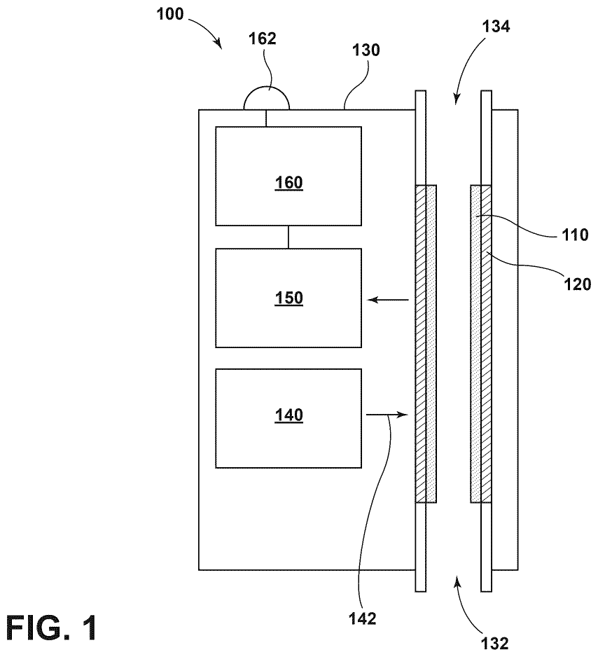

For purposes of description herein, the terms “upper,” “lower,” “right,” “left,” “rear,” “front,” “vertical,” “horizontal,” and derivatives thereof, shall relate to the disclosure as oriented in FIG. 1. Unless stated otherwise, the term “front” shall refer to a surface of the device closest to an intended viewer, and the term “rear” shall refer to a surface of the device furthest from the intended viewer. However, it is to be understood that the disclosure may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

The present disclosure relates to fluorescent sensor compounds that may be used to detect marijuana. In particular, these compounds may exhibit a fluorescence response when exposed to certain marijuana markers, including tetrahydrocannabinol (THC) and cannabidiol (CBD). Both THC and CBD may be present in the breath of a marijuana user at elevated levels for a time period after using marijuana. Therefore, fluorescent sensor compounds may be exposed to the breath of a subject, which may determine the presence of THC or CBD in breath. The magnitude of fluorescence response of these compounds may also be dependent on the concentration of THC or CBD in the breath. The concentration of THC and CBD in the breath may, in turn, be related to the length of time since the user has taken marijuana and the level of impairment of the user. Thus, the fluorescent sensor compounds described herein may provide information about the level of impairment of a user from taking marijuana. A level of THC in a user may correspond to psychological effects experienced by the user. The level of THC usually peaks in about 30 minutes after marijuana use and may diminish in 1-3 hours. A 3-hour window may be considered as the peak impairment window that is aligned with driving under the influence of a drug (DUID). After the peak time, THC may remain in the body as it is stored in fat cells for a much longer time. THC may remain detectable in saliva for up to 24 hours, in blood for 3 weeks, in urine for 1 month, and in hair for 3 months. Although testing of saliva, blood, urine, or hair may reveal that someone has recently used marijuana, this testing cannot determine whether the subject is currently under the influence. Therefore, detection technologies and methods based on testing of fluidic samples are not suited for roadside DUID testing or other scenarios where the goal is to determine whether the subject is under drug influence within the 3-hour window of impairment.

Fluorescent sensor compounds as related to the present disclosure may be capable of detecting THC and CBD with high sensitivity and selectivity. The fluorescent sensor compounds may be used to make sensors that may help law enforcement determine instantly if a subject is under the influence of marijuana. The sensors may also be referred to as fluorescent sensors or molecular sensors. The fluorescent sensor compounds described herein may detect THC, CBD, or both within the 3-hour impairment window, correlating the level of THC or CBD directly to the influence of the drug, or DUID. The methods of detection of the present disclosure may be both sensitive and selective in order to enable quantification of THC and CBD at trace levels in breath, and with minimal interference potentially caused by moisture and other species existing in the breath and local environment. In some examples, the average concentration of THC present in exhaled breath may be about 0.9 ng/L, about 0.14 ng/L, or about 0.05 ng/L, (corresponding to 007 ppb, 0.01 ppb, and 0.004 ppb) as measured in samples collected immediately after, about 1 hour after, or about 3 hours after smoking marijuana, respectively. Average tidal volume of human breath is 500-600 mL, while an expiratory reserve volume (ERV) is about 1100 ml for males and 800 mL for females. The concentration of THC detected in the breath may be correlated with THC levels found in the blood during the 3-hour period after marijuana use. The sensors may also be simple and quick to use, which is highly desirable for rapid onsite screening or monitoring of drug use. Additionally, the sensors may utilize noninvasive sampling, e g., from breath, rather than blood or urine. Compared to the current testing methods based on fluidic samples, breath analysis of THC may be an alternative approach for DUID testing for marijuana. In some examples, the sensors described herein may be in the form of a handheld breathalyzer-like device, which may be used for DUID testing for marijuana as simply as a current breathalyzer used for roadside DUI testing for alcohol. Other examples of handheld and/or small devices may be contemplated without departing from the teachings herein.

A unique class of fluorescent sensor compounds is described below. These compounds may selectively detect THC, as well as CBD, in gas phase with no, little, or controllable interference from common species existing in breath or environment, such as moisture (humidity), alcohol, acetone (as used in nail polish liquid), gasoline, cigarette smoke, perfumes, sun creams, among many others. Under elevated temperature, such as above 60° C., the limit of detection (LOD) as tested for THC and CBD may reach about 0.22 ppb for THC and about 0.04 ppb for CBD or about 5 pg for THC upon complete vaporization under high temperature e.g., 170° C. THC and CBD are nonvolatile compounds, with saturated vapor concentration under room temperature of 0.25 and 0.03 ppb, respectively. The melting points of THC and CBD are 64° C. and 68° C., respectively. Heating a sample above 60° C. may significantly increase the detection efficiency by maintaining more THC in the gas phase. In some examples, such heating may be provided by adding a heater in front of the detector, which may quickly heat a sample and vaporize THC and CBD before the markers reach the detector. LOD of sensors described herein may be sufficient for direct detection of THC and CBD in a breath sample within the impairment window after cannabis use. In further examples, a preconcentration device may be associated with breath sample collection. For example, a preconcentration membrane similar to membranes used for solid phase extraction (SPE) may be incorporated into a mouthpiece used for breath sample collection. Typical SPE membranes may concentrate chemical analytes by a factor of 1,000 or higher.

The fluorescent sensor compounds described herein may include rigid linear oligomers containing multiple aromatic rings, which may be an electron donor and an electron acceptor group, for example fluorene and pyridine thiazole groups respectively. These oligomers are electron donor-acceptor molecules in a conjugation structure, with an example of fluorene acting as donor and pyridine thiazole as acceptor. These molecules are strongly fluorescent in both solution and solid state. As such, other molecular moieties may be used in place of fluorene, including but not limited to groups that replace one or more of the carbon atoms in the fluorene group with a heteroatom, as long as the moiety is a fluorescent electron donor. Likewise, other molecular moieties may be used in place of pyridine thiazole, such as benzothiadiazole and including but not limited to groups that replace one or more of the carbon atoms in a benzothiadiazole group with a heteroatom, as long as the moiety is a fluorescent electron acceptor. For example, the fluorescence of these moieties, these compounds may be deposited in a thin film inside a quartz or glass tube, and may be fluorescent in this form. In this film example, upon interaction with THC or CBD, the fluorescence emission of the film may be effectively quenched due to the higher electron density on the main chain when the molecules interact with the aromatic groups of the THC/CBD with increased dipole-dipole interactions. The extent of the emission quenching may be measured and correlated to the gas phase concentration of THC. A calibration curve may be established and then used to determine gas phase concentration of THC based on measured emission quenching.

An example sensor with a film of the fluorescent sensor compound was made and tested against potential interferents. Moisture and many common chemicals, such as alcohol, acetone, and gasolines, may cause opposite sensor response compared to THC and CBD, i.e., an increase in emission intensity, without wishing to be bound by theory, is likely due to the non-specific swelling mechanism, meaning that the temporary reduction of the intermolecular dipole-dipole interactions enhances the emission. Nonetheless, the responses obtained for these interferents were minimal. For example, when tested under 54% relative humidity (equivalent to water vapor concentration of 12,455 ppm) the sensor response observed in this example was only +0.044%, compared to the −0.27% response obtained for THC tested under only 396 ppb (at 75° C.), which represents a concentration level about 5 orders of magnitude lower than that of moisture. When exposed to 72.5 ppm ethanol, a common substance present in breath after drinking, the sensor generated only +0.019% response in this example, whereas a much larger response (though in the opposite direction), −0.27%, was obtained for THC, though tested under a much lower concentration, 39.6 ppb (at 75° C.). Besides, the fluorescence responses of interferents are not only in the opposite direction, but also reversible and quick (i.e., recovered in a short period of time), which can make the irreversible, relatively slower fluorescence quenching (decrease) responses towards THC and CBD easy to distinguish in the practical tests. The quick, reversible response of the interferents would not affect the quantification measurement of THC or CBD. Detailed results of the sensor testing are presented herein.

Compared to current chemical sensors and analytical instrumentation, like chromatography coupled with mass spectrometry, the current disclosed sensors and methods may provide on-site detection of marijuana use. The sensors may have high sensitivity, with LOD down to low parts per billion (ppb) for gas phase or low picogram (pg) for surface detection. It may then be possible to develop the sensor into a breathalyzer-like detector to serve law enforcement and other customers. In other examples, it may be possible to couple the sensor with a solid phase extraction component, such as an extraction membrane in a mouthpiece. The addition of an extraction membrane may allow THC and CBD to be effectively concentrated on the extraction membrane, which would allow for detection of THC and CBD directly from a surface, and may further increase the detection capability by lowering the LOD of breath analysis by two or more orders of magnitude. The sensors may also have high selectivity, minimizing false positives in detection, especially against common interferents present in breath and the local environment. The sensors may be easy to use, low cost, stable, and suited for quick onsite detection.

Turning now to the structure of the fluorescent sensor compounds, in some examples a fluorescent sensor compound may include an oligomer having a general structure of

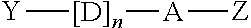

Generally, group D may be any electron donor group that is fluorescent while group A may be any electron acceptor group that is fluorescent. In some examples, the structure may include D as an electron donor group according to Formula (I):

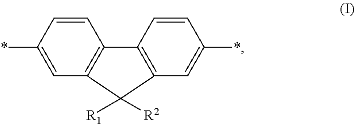

In other examples, the structure may also include a group A, which is an electron acceptor group according to Formula (II):

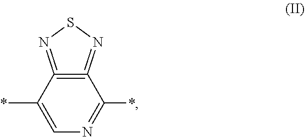

In the formulas for functional groups described herein, such as D and A above, a solid line terminating in a dot represents a bonding location where the functional group may bond to another atom or another functional group. The D and A groups each have two of these bonding locations, which means that each D and A group may bond with two other atoms or functional groups. The fluorescent sensor compounds described herein may include a linear oligomer that includes one or more of each of the D and A groups. In Formula (I), the groups R₁ and R₂ may be the same or may be different. In some examples, the groups R₁ and R₂ may be an alkyl chain. If the compound includes multiple D groups, then the R₁ and R₂ groups may be different in different respective D groups. In Formula (II), the A is a pyridine thiazole group. Additionally, in the general structure Y-[D]ₙ-A-Z, Y may be R₃, R₃-A, or R₃-D-A. The Z group may be R₃ or [D]ₘ-R₃. In the Y and Z groups, R₃ may be an alkoxyphenyl end group. The subscripts n and m can be integers indicating a number of D groups bonded together linearly, where n is an integer from 1 to 6 and m is an integer from 1 to 4. In various examples, the fluorescent sensor compound may include a total number of A groups from 1 to 4, and a total number of D groups from 1 to 12. In certain examples, the fluorescent sensor compound may include a total number of A groups from 1 to 2 and a total number of D groups from 1 to 5.

Consistent with the above general formula, several more specific oligomer structures are shown in Formulas (III) through (X). In some examples, the fluorescent sensor compound can include an oligomer according to one of the formulas:

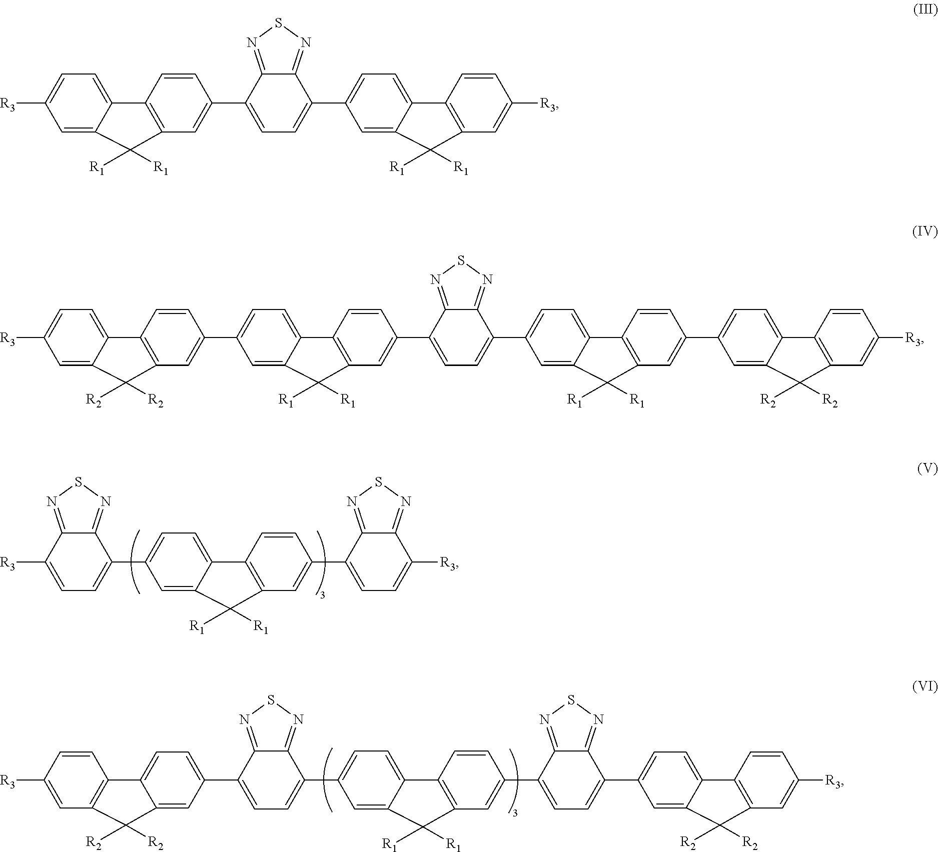

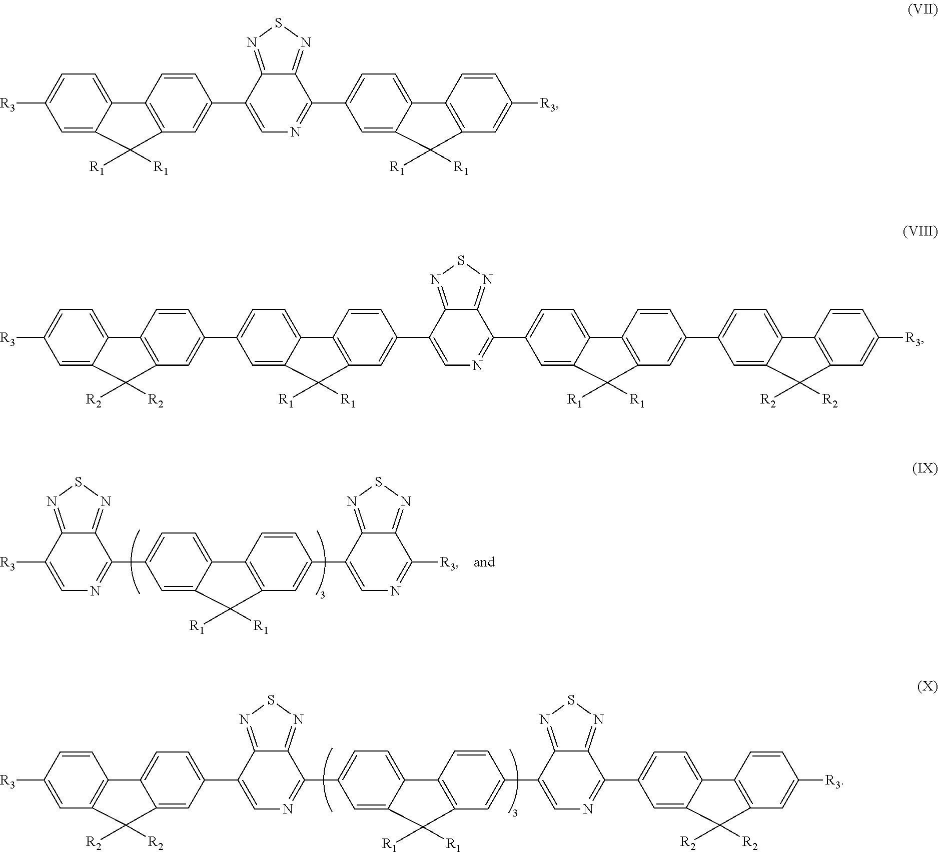

In these formulas, R₁ and R₃ can be defined in the same way as in Formulas (I) and (II) above. The R₂ group can be an alkyl group similar to R₁. In some examples R₁ and R₂ may be the same and may be an alkyl group, in other examples R₁ and R₂ or different and may be different alkyl groups.

In certain examples, R₁ and R₂ may be independently selected from the group consisting of —CH₃, —C₂H₅, —C₃H₇, —C₄H₉, —C₅H₁₁, C₆H₁₃, C₇H₁₅, and C₈H₁₇. In some examples, the R₁ and R₂ groups can be linear alkyl groups. In other examples, the R₁ and R₂ may be independently linear alkyl chains having from 1 to 8 carbon atoms.

As mentioned above, the R₃ group may be an alkoxyphenyl end group. The alkoxyphenyl end group may include a phenyl group and an alkoxy group. The phenyl group may be directly bonded to the remainder of the oligomer, and the alkoxy group can be bonded to the phenyl group. In some examples, the alkoxy group may be bonded to the phenyl group at the para-position. The alkoxy group may include a variety of linear alkoxy groups or branched alkoxy groups. In some examples, the alkoxy group may include an oxygen atom and from 1 to 10 carbon atoms. In further examples, the alkoxy group may include an oxygen atom and from 1 to 6 carbon atoms or from 1 to 5 carbon atoms. Specific examples of alkoxy groups are shown in Formulas XI through XX below:

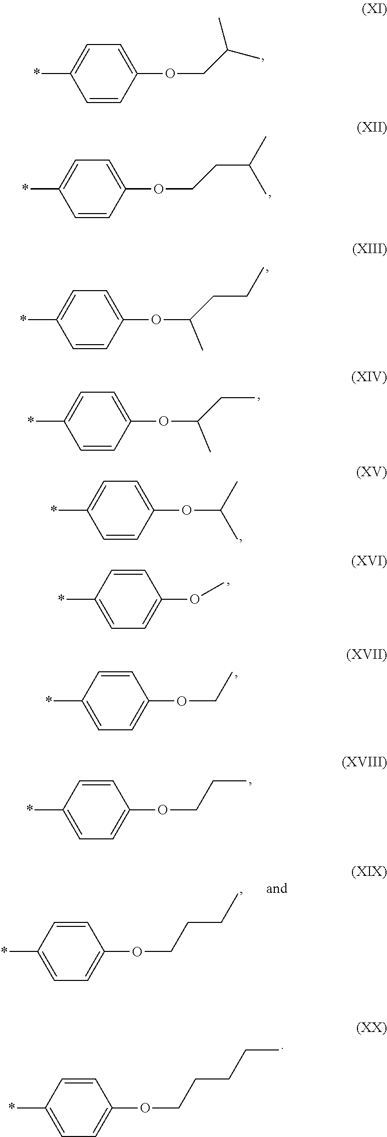

According to further methods of the present disclosure, the fluorescent sensor compounds may fluoresce when exposed to light. In some examples, the fluorescent sensor compounds can fluoresce when exposed to ultraviolet light, in various examples, the ultraviolet light may have a wavelength of 100 nm to 400 nm. The ultraviolet light may be UVA (315-400 nm), UVB (280-315 nm), or UVC (100-280 nm). When the fluorescent sensor compounds are exposed to THC or CBD, the fluorescence intensity may decrease. The sensitivity of the fluorescent sensor compound may depend on the magnitude of fluorescence intensity change when exposed to a given concentration of THC or CBD. In some examples, THC molecules may bind to the fluorescent sensor compound molecules. Without wishing to be bound by theory, the binding affinity may be affected by T-T interactions between the aromatic ring of THC and the fluorescent sensor molecule, and also by dipole-dipole interactions between THC and the fluorescent sensor molecule. The dipole-dipole interactions may be strengthened by designing the fluorescent sensor compound with electron donor and electron acceptor groups as explained above. Larger numbers of electron donor groups and electron acceptor groups may provide greater sensitivity due to stronger interactions between THC and the fluorescent sensor compound. In some examples, the fluorescent sensor compound may have from 1 to 5 electron donor groups and from 1 to 5 electron acceptor groups. In further examples, the fluorescent sensor compound can have from 1 to 2 electron acceptor groups, from 1 to 4 electron donor groups, from 1 to 3 electron donor groups, or from 1 to 2 electron donor groups.

The higher fluorescence intensity response may also allow for higher sensitivity in some examples. In various examples, all the electron acceptor groups in the fluorescent sensor compound may be benzothiadiazole or pyridine thiazole or any other aromatic electron acceptor group.

The fluorescent sensor compounds may be described as oligomers, as opposed to polymers, because the compounds may be made up of a relatively small number of functional groups. In some examples, the fluorescent sensor compound may consist of 4 to 12 functional groups, where the functional groups include electron donor groups, electron acceptor groups, and end groups. The electron donor groups may be the fluorene groups as described above. The electron acceptor groups may be benzothiadiazole as described above. The end groups may be alkoxyphenyl groups as described above. In further examples, the total number of these functional groups in the fluorescent sensor compound can be from 4 to 10, 4 to 9, 5 to 9, 5 to 8, 5 to 7, 5 to 6, or 7 to 9. In further examples, the fluorescent sensor compound can have a molecular weight from about 700 g/mol to about 2500 g/mol.

### Sensor Compound Morphology

In some examples, the fluorescent sensor compounds may form microstructures or nanostructures such as nanofibrils or nanoparticles. In certain examples, the nanostructures may form partially through π-π interactions between the aromatic groups of adjacent molecules of the fluorescent sensor compound. A sufficient amount of the fluorescent sensor compound may form porous structures made up of these nanostructures. In some examples, nanofibrils may form a porous net structure. This structure may be useful for detecting THC and CBD in the gas phase, because THC and CBD molecules can penetrate into the net structure. Additionally, the net structure may have a high surface area, which increases the likelihood that a THC or CBD molecule will interact with the fluorescent sensor compound. However, if the porous net structure is formed as a thick layer, then at some depth within the layer it may become difficult to detect changes in fluorescence caused by THC or CBD molecules. For example, fluorescence changes of nanofibrils of the fluorescent sensor molecules deep in the film may be obscured by other nanofibrils closer to the surface. Therefore, above a certain threshold thickness it may not be useful to increase the thickness of a porous film of the fluorescent sensor compound. In some examples, a porous film of the fluorescent sensor compound can have a thickness from about 1 μm to about 100 μm, or from about 10 μm to about 100 μm, or from about 50 μm to about 100 μm, or from about 1 μm to about 10 μm, or from about 1 μm to about 50 μm.

In other examples, the fluorescent sensor compound can be in the form of a thin, non-porous film. The thin, non-porous film can have a thickness from about 100 nm to about 10 μm, or from about 100 nm to about 5 μm, or from about 100 nm to about 1 μm, or from about 100 nm to about 500 nm, or from about 500 nm to about 1 μm, or from about 500 nm to about 5 μm, or from about 500 nm to about 10 μm, or from about 1 μm to about 5 μm, or from about 1 μm to about 10 μm, or from about 5 μm to about 10 μm. Whether the fluorescent sensor compound is deposited as a porous film or as a non-porous film, the compound can be deposited on a substrate. Substrates may include, but are not limited to, glass, silicon, and quartz. The substrate may have a variety of shapes, such as a flat plate, a pair of flat surfaces that form a channel, a tube, baffles, a screen or grate, or other shapes. In some examples, the substrate and the film of the fluorescent sensor compound may be designed to provide contact between the fluorescent sensor compound and a flow of exhaled breath or other gas.

### Fluorescent Sensors for Detecting Marijuana

The present disclosure also describes a fluorescent sensor for detecting marijuana. The fluorescent sensors can include a fluorescent sensor compound as described above. The sensor may also include a fluorescence detector oriented to detect a fluorescence response from the fluorescent sensor compound. In some examples, the fluorescence response may be a reduction in fluorescence intensity upon exposure to THC or CBD.

Referring to FIG. 1 for a schematic diagram of an example fluorescent sensor 100. This sensor includes a porous film 110 of a fluorescent sensor compound on a substrate 120. In this example, the substrate is a tube (cross-section of the tube is shown) and the film of the fluorescent sensor compound is deposited on the inside surface of the tube. A cross-section of the tube is shown in the FIG. 1. The sensor also includes a housing 130. The housing includes a breath inlet 132 and an outlet 134. In this example, the housing may be shaped as a small rectangular box with a breath inlet hole for a user to breath into and a breath outlet hole for the breath to exit after passing through or over the sensor. The breath inlet and outlet are connected to the tube containing the fluorescent sensor compound so that exhaled breath can pass the film of fluorescent sensor compound. The sensor also includes an excitation light source 140 and a fluorescence detector 150. In this example, the tube substrate is transparent so that light 142 can be pass through the tube. The light from the excitation light source can pass through the tube to excite the fluorescent sensor compound. Light produced by fluorescence can also pass through the tube to be detected by the detector. A signal from the detector is sent to a comparator 160. The comparator is also connected to an indicator 162. Connections between the comparator and the fluorescence detector and the indicator are shown as solid lines. The comparator may be configured to compare the fluorescence intensity detected by the detector with a threshold value, and if the fluorescence intensity falls below the threshold upon exposure of the sensor to a sample then this may suggest that the sample contains THC or CBD. The comparator can produce a signal using the indicator 162 if THC or CBD is detected. The indicator 162 may include a visible indicator, such as an LED light, or an audible indicator such as an alarm, buzzer, or beeper. In alternative examples, the detector can be connected to an external module such as a personal computer, which can display test results in a variety of ways.

The example shown in FIG. 1 may be designed to directly detect THC or CBD in exhaled breath. In other examples, the sensor can include a condensing surface. The sensor housing can direct exhaled breath to the condensing surface to form a breath condensate on the condensing surface. The breath condensate can then be heated to evaporate the breath condensate. The vapor can be directed to contact the fluorescent sensor compound to detect THC or CBD in the vapor. These compounds may be more concentrated in the breath condensate compared to freshly exhaled breath. Therefore, in some cases, the compounds can be detected with a greater sensitivity when the breath is condensed in this way. In some examples, the sensor can include a heater to heat the breath condensate. In other examples, a two-part sensor can be made, in which a user breathes into one part containing a condensing surface to collect a breath condensate on the condensing surface, and then the condensing surface can be loaded or connected to a second part of the sensor, where the second part includes the fluorescent sensing compound for detecting THC or CBD. In certain examples, the condensing surface can be incorporated in a disposable cartridge for one-time use. The condensing surface can be a pre-concentration membrane similar to membranes used for solid-phase extraction. The membrane can be configured to interact with THC and/or CBD so that these compounds are captured from the exhaled breath.

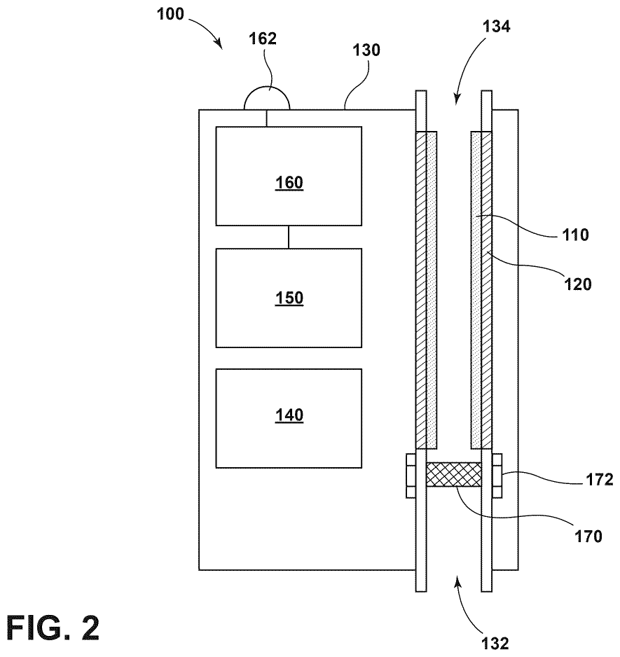

Referring to FIG. 2 for a schematic diagram of a different example fluorescent sensor 100. This example includes a housing 130 with a breath inlet 132 and breath outlet 134. The breath inlet leads to a condensing surface 170, which in this example is a pre-concentration membrane. The breath passes through the pre-concentration membrane and THC and CBD in the breath are captured by the membrane. After a user has breathed into the sensor, a heater 172 can be used to heat the membrane and vaporize the condensed components of the breath. In this example, the heater is an electric heating element shaped as a ring surrounding the pre-concentration membrane (a cross-section of the ring-shaped heating element is shown). The vaporized material can then contact the film of fluorescent sensor compound 110 on the substrate 120 as in the previous example. In some examples, the vaporized material can diffuse to the film of fluorescent sensor compound, and in other examples, the sensor can include a pump to pump the vaporized material to the film of fluorescent sensor compound. This example also includes an excitation light source 140, a fluorescence detector 150, a comparator 160, and an indicator 162, as in the previous example, all of which may provide similar functions.

In some examples, the sensors described herein can detect THC or CBD from exhaled breath containing the THC or CBD at concentrations with a limit of detection from about 0.0008 parts per billion by volume (ppbv) to about 0.2 ppbv. In certain examples, the sensor can utilize a condensing surface to condense and pre-concentrate compounds from the exhaled breath. This may increase the sensitivity of detection. In some such examples, the limit of detection can be from about 0.0008 ppbv to about 0.08 ppbv. In other examples, the sensor can detect THC or CBD directly from exhaled breath. In some such examples, the limit of detection can be from about 0.02 ppbv to about 0.2 ppbv. In other examples, typical concentrations of THC in the breath of a user within three hours after using marijuana can range from about 0.004 ppbv to about 0.07 ppbv in some examples. The total amount of THC or CBD present in the sensor during detection can be on the order of nanograms (ng) to picograms (pg). In some examples, the sensor may have a limit of detection based on a total amount of THC or CBD inside the sensor, where the limit of detection is from 1 pg to 100 ng, or from 10 pg to 10 ng, 100 pg to 10 ng, 500 pg to 10 ng, 1 ng to 10 ng, 1 pg to 1 ng, 1 pg to 500 pg, 1 pg to 100 pg, or 10 pg to 100 pg.

The sensors described herein may be reusable or may be manufactured to be disposable. When configured to be disposable, the sensor may be made of inexpensive materials, including some or all of the materials being biodegradable. Further, only parts of the sensor may be configured to be disposable on an overall reusable sensor. For example, the member may be disposable while the housing and other components may be reusable with other disposable membranes.

### Methods of Detecting Marijuana Breath Markers

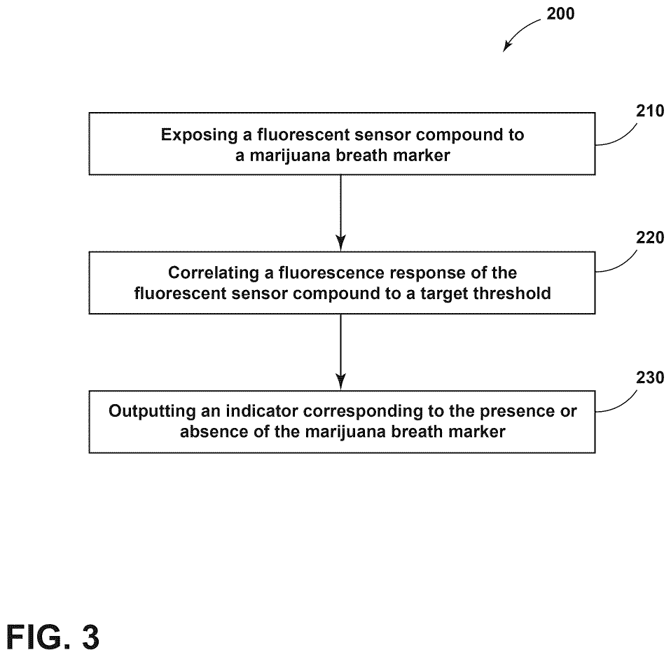

The present disclosure also describes methods of detecting marijuana breath markers, in some examples, the marijuana breath marker can be THC or CBD. Referring to FIG. 3 for a flowchart illustrating one example method 200 of detecting a marijuana breath marker. The method includes: exposing a fluorescent sensor compound to a marijuana breath marker 210, correlating a fluorescence response of the fluorescent sensor compound to a target threshold 220, and outputting an indicator corresponding to the presence or absence of the marijuana breath marker 230. In some examples, the breath sample may be directly exhaled breath, a breath condensate, a pre-concentrated breath, or a combination thereof.

In some examples, the fluorescence response to THC or CBD can include a drop in fluorescence intensity upon exposure to the THC or CBD. The fluorescence intensity dropping below a target threshold can indicate the presence of THC or CBD. The target threshold can be determined as a percentage drop based on a baseline fluorescence intensity. In some examples, a sensor can be pre-tested with known samples of THC or CBD to determine a threshold fluorescence response that has a sufficient likelihood of indicating the presence of THC or CBD, as opposed to background noise. In certain examples, the threshold fluorescence response can be selected to represent a 95% likelihood that THC or CBD is present, or a 99% likelihood, or a 99.9% likelihood. The sensor may be capable of detecting very small drops in fluorescence intensity, and therefore may be capable of accurately detecting small concentrations of THC and CBD. In some examples, the threshold drop in fluorescence intensity to indicate the presence of THC or CBD can be from about 0.02% to about 1%, or from about 0.02% to about 0.5%, or from about 0.02% to about 0.2%, or from about 0.02% to about 0.1%, or from about 0.02% to about 0.05%, or from about 0.05% to about 1%, or from about 0.05% to about 0.5%, or from about 0.05% to about 0.2%, or from about 0.05% to about 0.1%, or from about 0, 1% to about 1%, or from about 0.1% to about 0.5%, or from about 0.1% to about 0.2%.

### Synthesis Examples

The synthesis of the analogous compounds in as in formula (III), (IV), and (V) is described in PCT Application No. WO2024215483A1, which is herein incorporated by reference in its entirety.

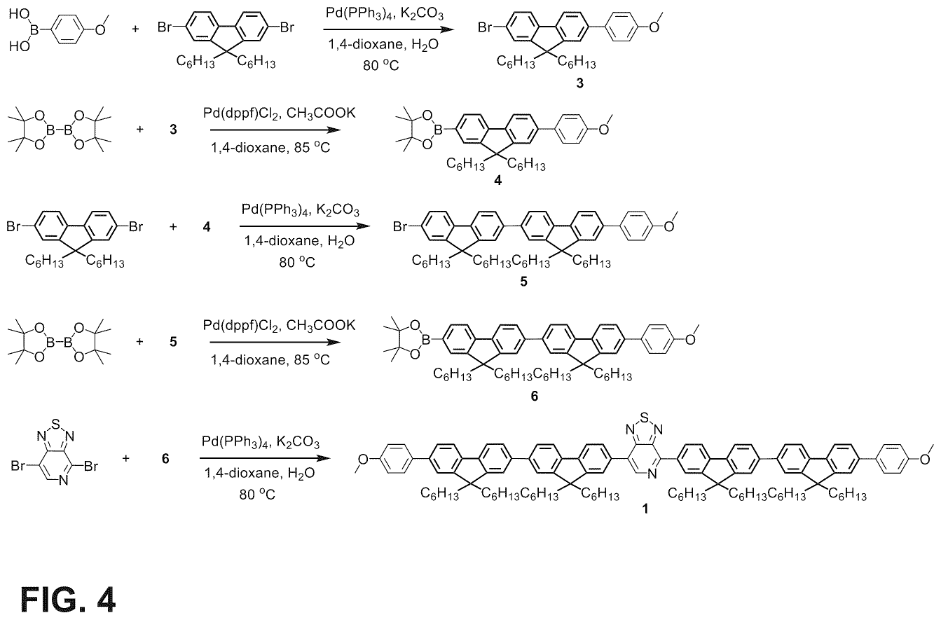

With reference to FIG. 4, synthesis of the analogous compound as in Formula (VIII) can be achieved using the following.

2-bromo-9,9-diethyl-7-(4-methoxyphenyl)-9H-fluorene (3). To a stirred solution of (4-methoxyphenyl) boronic acid (1 g, 6.57 mmol), 2,7-dibromo-9,9-diethyl-9H-fluorene (3.8 g, 7.9 mmol), and tetrakis(triphenylphosphine)palladium (Pd(PPh₃)₄) (380 mg, 0.33 mmol) in deoxygenated 1,4-dioxane (40 mL), an aqueous solution of potassium carbonate (K₂CO₃) (8 mL, 4.0 M) was added. Then the reaction mixture was heated to 80° C. and kept under an argon atmosphere overnight. The solvent was then evaporated under reduced pressure. The resulting residue was mixed with water (40 mL) and extracted with dichloromethane (3×40 mL). The combined organic solutions were washed with saturated brine, dried over sodium sulfate (Na₂SO₄), and concentrated under reduced pressure. The final residue was then subjected to column chromatography on silica gel (petroleum ether:dichloromethane=20:1 as the eluent) to obtain compound 3 (2.73 g, 5.26 mmol, 79% yield).

2-(9,9-diethyl-7-(4-methoxyphenyl)-9H-fluoren-2-yl)-4,4,5,5-tetramethyl-1,3,2 dioxaborolane (4). A mixture of 3, synthesis described above, (2.73 g, 5.26 mmol), bis(pinacolato)diboron (2.68 g, 10.52 mmol), potassium acetate (CH₃COOK) (2.58 g, 26.4 mmol), and dichloro[1,1′-bis(diphenylphosphino) ferrocene]palladium(II) (Pd(dppf)Cl₂) (0.39 g, 0.53 mmol) in deoxygenated 1,4-dioxane (40 mL) was stirred at 85° C. under argon for 8 h. After removal of the solvent under vacuum, the residue was poured into water (40 mL) and extracted with ethyl acetate (3×45 mL). The combined organic layer was washed with saturated brine, dried over Na₂SO₄, and then concentrated under vacuum. The residue was purified by column chromatography on silica gel (petroleum:dichloromethane=5:1 as the eluent) to obtain compound 4 (2.07 g, 3.65 mmol, 69% yield).

7-bromo-9′,9′-diethyl-9,9-dihexyl-7′-(4-methoxyphenyl)-9H,9′H-2,2′-bifluorene (5). To a stirred solution of 4, synthesis described above, (2.07 g, 3.65 mmol), 2,7-dibromo-9,9-dihexyl-9H-fluorene (2.18 g, 4.43 mmol), and Pd(PPh₃)₄ (213 mg, 0.18 mmol) in deoxygenated 1,4-dioxane (40 mL), an aqueous solution of potassium carbonate (8 mL, 4.0 M) was added. Then the mixture was heated to 80° C. and stirred under argon overnight. The solvent was evaporated under vacuum. The residue was poured into water (40 mL) and extracted with dichloromethane three times using 40 ml of dichloromethane with each extraction. The combined organic layer was washed with saturated brine, dried over Na₂SO₄, and then concentrated under vacuum. The residue was purified by column chromatography on silica gel (petroleum:dichloromethane=15:1 as the eluent) to obtain compound 5 (2.14 g, 2.52 mmol, 68% yield).

2-(9,9-dihexyl-7-(4-methoxyphenyl)-9H-fluoren-2-yl)-4,4,5,5-tetramethyl-1,3,2-dioxaborolane (6). A mixture of 5, synthesis described above, (2.14 g, 2.52 mmol), bis(pinacolato)diboron (0.99 g, 3.96 mmol), potassium acetate (0.78 g, 7.9 mmol), and Pd(dppf)Cl₂ (0.1 g, 0.14 mmol) in deoxygenated 1,4-dioxane (30 mL) was stirred at 85° C. under argon for 8 h. After removal of the solvent under vacuum, the residue was poured into water (40 mL) and extracted with ethyl acetate three times using 45 ml of ethyl acetate with each extraction. The combined organic layer was washed with saturated brine saturated, dried over Na₂SO₄, and then concentrated under vacuum. The residue was purified by column chromatography on silica gel (petroleum:dichloromethane=5:1 as the eluent) to obtain 6 (1.36 g, 1.51 mmol, 60% yield).

4,7-bis(9,9,9′,9′-tetrahexyl-7′-(4-methoxyphenyl)-9H,9′H-[2,2′-bifluoren]-7-yl)-[1,2,5]thiadiazolo[3,4-c]pyridine (1). To a stirred solution of 4,7-dibromo-[1,2,5]thiadiazolo[3,4-c]pyridine (2.06 g, 0.7 mmol), 6, synthesis described above, (1.36 g, 1.51 mmol), and Pd(PPh₃)₄ (140 mg, 0.12 mmol) in deoxygenated 1,4 dioxane (40 mL), an aqueous solution of potassium carbonate (8 mL, 4.0 M) was added. Then the mixture was heated to 80° C. and stirred overnight under argon. The solvent was evaporated under vacuum. The residue was poured into water (40 mL) and extracted with dichloromethane three times using 40 mL of dichloromethane with each extraction. The combined organic layer was washed with saturated brine, dried over Na₂SO₄, and concentrated under vacuum. The residue was purified by column chromatography on silica gel (petroleum:dichloromethane=2:1 as the eluent) to afford molecule 1 (0.68 g, 0.41 mmol, 59% yield).

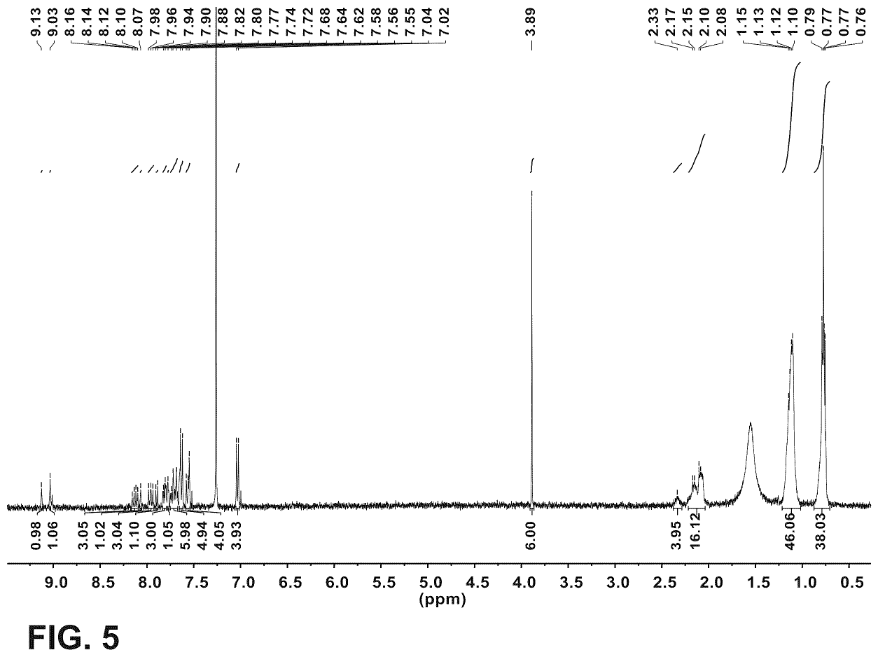

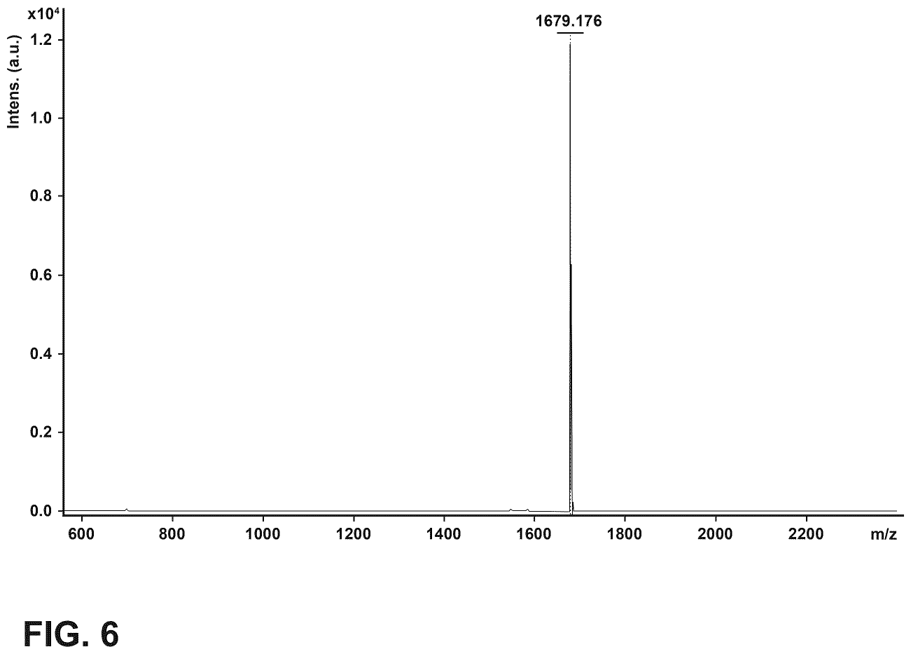

The resulting target molecule 1 was confirmed by ¹H NMR and MALDI-MS as below and in FIGS. 5 and 6, respectively.

Molecule 1. ¹H NMR (400 MHz, Chloroform-d, ppm) δ 9.13 (s, 1H), 9.03 (s, 1H), 8.13 (dd, J=15.2, 7.8 Hz, 3H), 8.07 (s, 1H), 7.99-7.92 (m, 3H), 7.89 (d, J=7.6 Hz, 1H), 7.81 (d, J=7.9 Hz, 3H), 7.77 (s, 1H), 7.75-7.67 (m, 6H), 7.63 (d, J=8.8 Hz, 5H), 7.58-7.54 (m, 4H), 7.03 (d, J=8.7 Hz, 4H), 3.89 (s, 6H), 2.33 (s, 4H), 2.13 (dd, J=26.5, 7.2 Hz, 16H), 1.12 (dd, J=11.6, 5.1 Hz, 46H), 0.87-0.71 (m, 38H). MALDI-TOF-MS: (m/z)=1679.176.

### Fabrication of Microribbons of Molecule 1 Via Living Seeded Self-Assembly

Microribbons of 1 were first generated by injecting 1 ml of acetonitrile into 0.2 mL of a chloroform solution of 1 (0.5 mg/ml) in a 4 mL vial. The mixture was then allowed to age at 25° C. for 48 h. Following the removal of the supernatant from the hexagonal platelets suspension, 2 ml of acetonitrile was added. The resulting solution containing the microribbons was then subjected to sonication at −35° C. for 2 min to obtain the seeds of 1 (0.05 mg/ml). By adding varying volumes (0.07, 0.02, 0.01, 0.005 ml) of an acetonitrile solution containing the seeds of 1 (0.05 mg/ml) to 1.1 ml of the supersaturated solution of 1 (0.01 mg/ml) in chloroform/acetonitrile mixtures (v/v, 1/10) in a vial and aging at 25° C. for 30 min, the microribbons with controlled sizes were successfully fabricated.

### Examples

Two molecules were tested as fluorescent sensors towards THC and CBD:

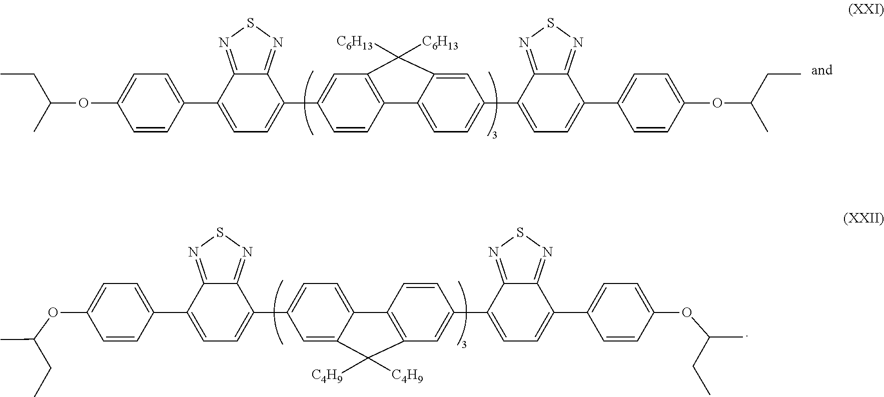

The molecule of formula (XXI) may also be referred to as TFT-C6, using IUPAC naming is called 7,7′-(9,9,9′,9′,9″,9″-hexahexyl-9H,9r H,9″H-[2,2′:7′,2″-terfluorene]-7,7″-diyl)bis(4-(4-sec-butoxy)phenyl) benzo[c][1,2,5]thiadiazole). The molecule of formula (XXII) may also be referred to as TFT-C4. Note that the side group attached to the fluorene moiety may be adjusted in different lengths of alkyl chains, ranging from C₂ to C₈. Additionally, the benzothiadiazole moieties in both TFT-C6 and TFT-C4 may be replaced with pyridine thiadiazole. As currently tested, both C₆ (TFT-C6) and C4 (TFT-C4) substituted molecules as fabricated as thin film, demonstrate effective fluorescent sensor response towards THC and CBD.

Results of gas phase detection of THC and CBD, and potential interferents present in breath and local environment were tested. First, the TFT-C6 molecules were caused to self-assemble by the following procedure. First, a 0.1 mL CHCl₃ solution with TFT-C6 (6 mg/ml) was injected into a vial. Then 2 mL methanol was added to the same vial aging for 4 h. The resulting assemblies will be suspended in the solutions for casting inside the quartz tubes. A similar self-assembly methodology for molecule 1 can be found above.

Fluorescent sensing was then performed using the following procedure. A 10 μl of the aggregates suspension in methanol was cast inside a quartz tube about 0.3 cm away from the air-inlet. The remaining solvent was removed from the quartz tube by a capillary. The tube was then dried by a blower for 30 s at first and then in vacuum for 2 h to remove all the solvent inside. A 0.2 mL THC or CBD methanol solution (1 mg/mL) was transferred into a 40 mL vial, followed by drying under vacuum for 4 h to remove the solvent. The vial was placed in a clean oven at different temperatures for 30 min to get the saturated vapor inside the vial. The headspace sample of the vial was then pumped into the sensor tube (at flow rate of 150 sccm) for 10 s for sensor testing.

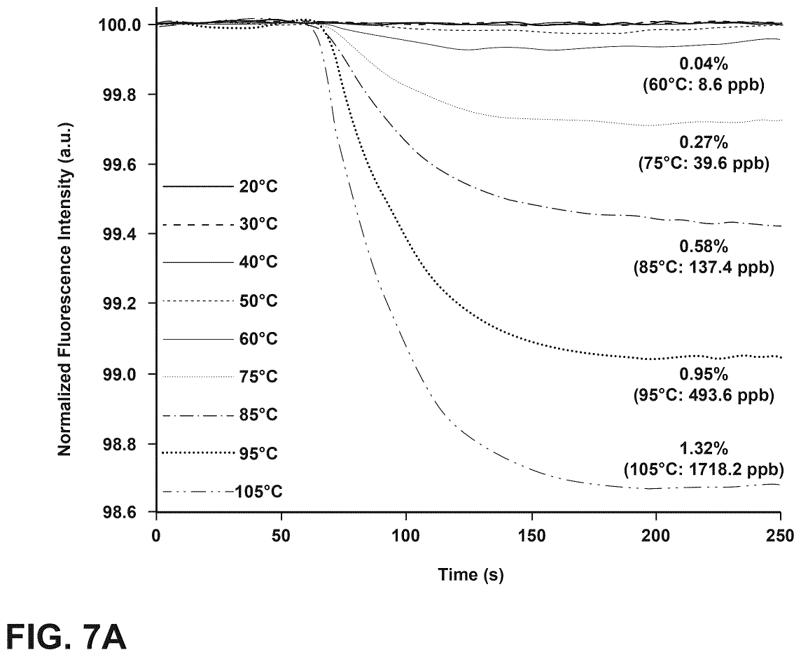

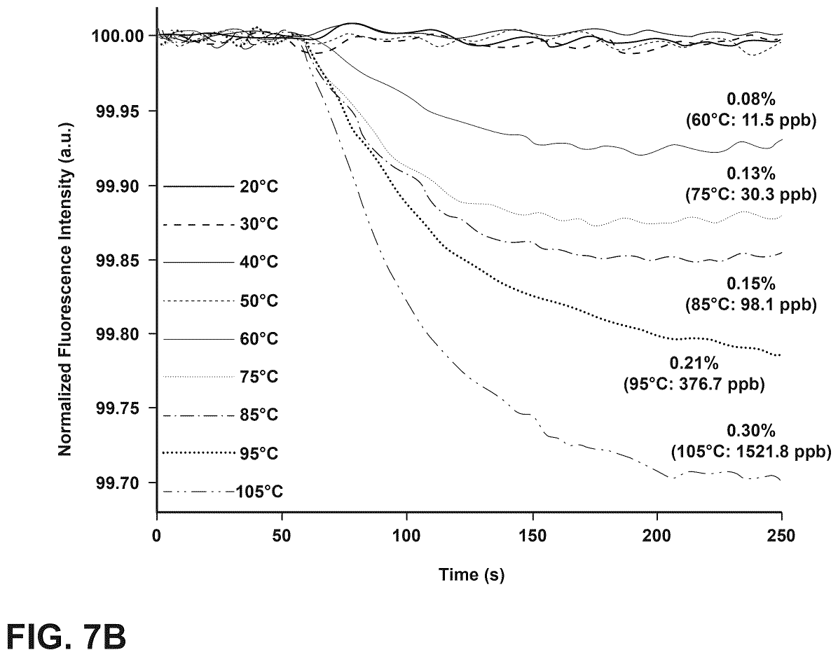

Referring to FIG. 7A, a fluorescence quenching response is shown to THC vapor at different temperatures. FIG. 7B shows fluorescence response to CBD vapor at different temperatures. The predicted vapor pressure of THC under different temperatures were: 60° C.: 8.6 ppb, 75° C.: 39.6 ppb, 85° C.; 137.4 ppb, 95° C.: 493.6 ppb, 105° C.: 1718.2 ppb, predicted vapor pressure of CBD under different temperatures: 60° C.: 11.5 ppb, 75° C.: 30.3 ppb, 85° C.: 98.1 ppb, 95° C.: 376.7 ppb, 105° C.: 1521.8 ppb.

The limit of detection (LOD) was determined by the following equations (i) and (ii) (500 points were chosen to calculate the root-mean-square value of the noise: RMS_{Noise}):

$$LOD = \frac{ak {( 3 \times {RMS}_{Noise} )}^{n}}{1 + {k ( 3 \times {RMS}_{Noise} )}^{n}} \qquad ( i )$$

$${RMS}_{Noise} = \sqrt{\frac{\sum {( {I}_{i} - {I}_{c} )}^{2}}{N \times {( {I}_{0} )}^{2}}} \qquad ( ii )$$

where Iᵢ and I_{c} represent experimental and corrected fluorescence intensity values, respectively, Iₒ is the fluorescence intensity value when time is 0, n represents the number of data points (500 points were chosen to calculate in the current example).

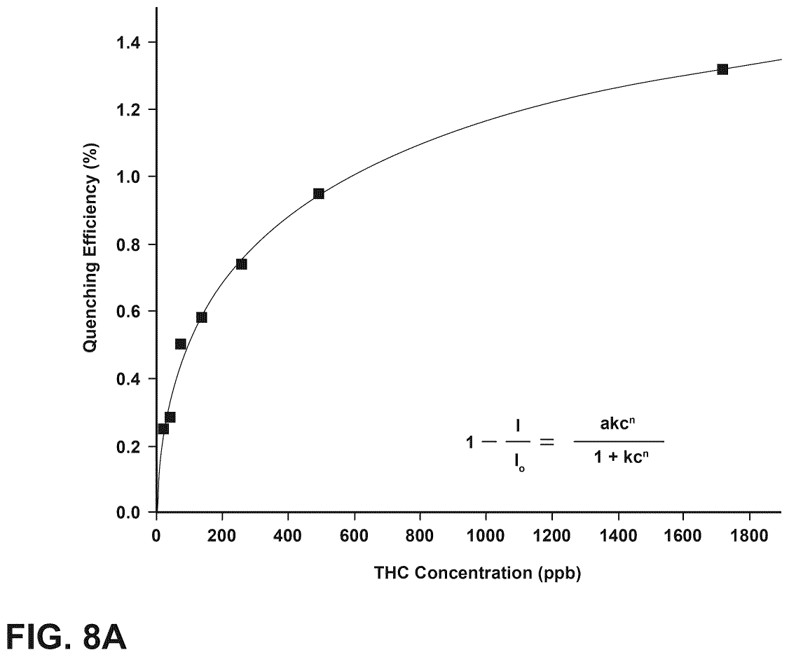

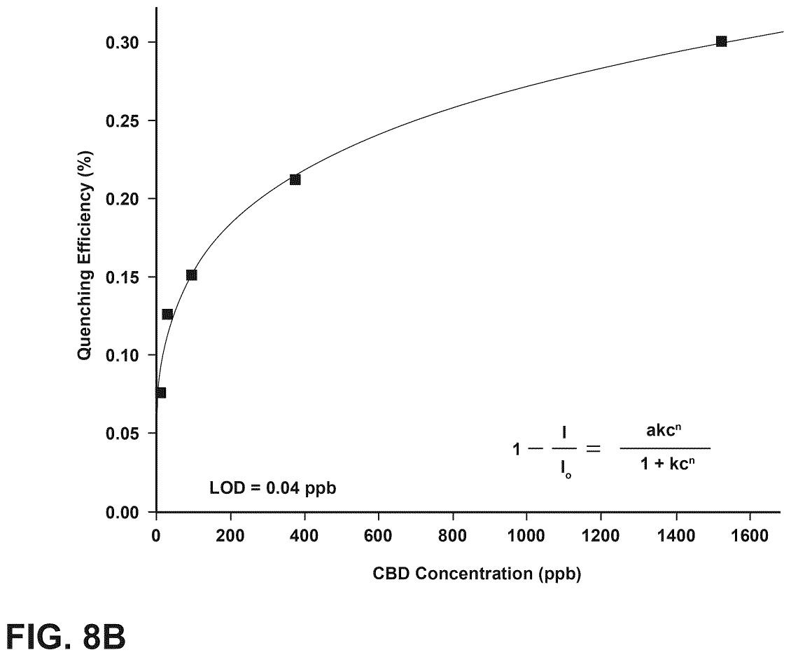

FIGS. 8A-8B show fitting curves and equation of the fluorescence quenching response obtained for THC (FIG. 8A) and CBD (FIG. 8B) at different vapor pressures (controlled by temperatures). The exponent “n” is a fitting parameter indicating the adjustment of Langmuir adsorption model due to the deviation from the ideal complete monolayer adsorption on the surface. The calculated LOD of THC and CBD are 0.22 ppb and 0.04 ppb, respectively.

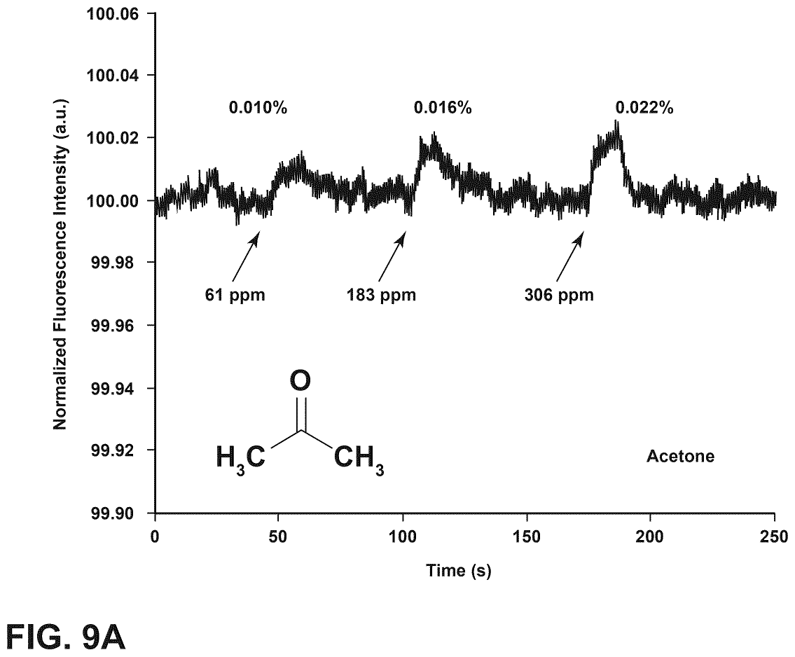

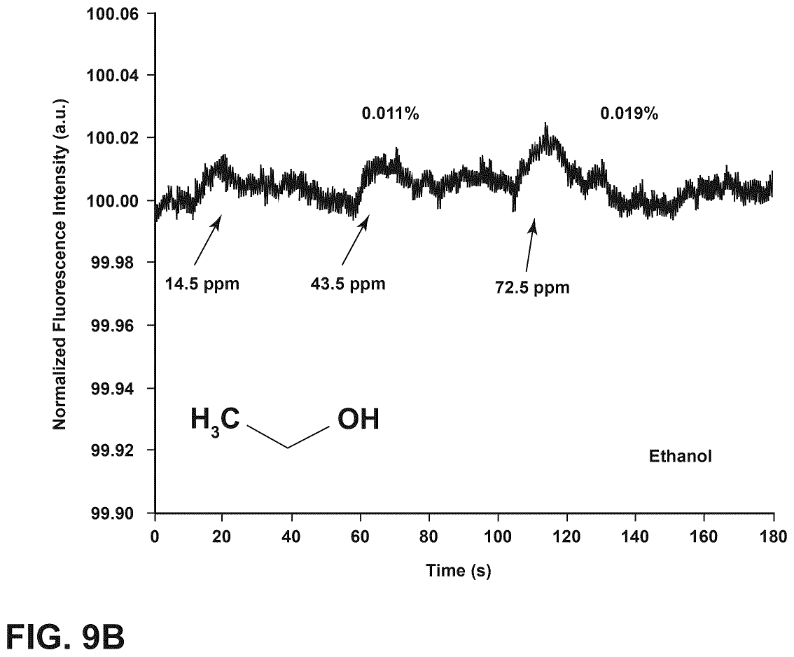

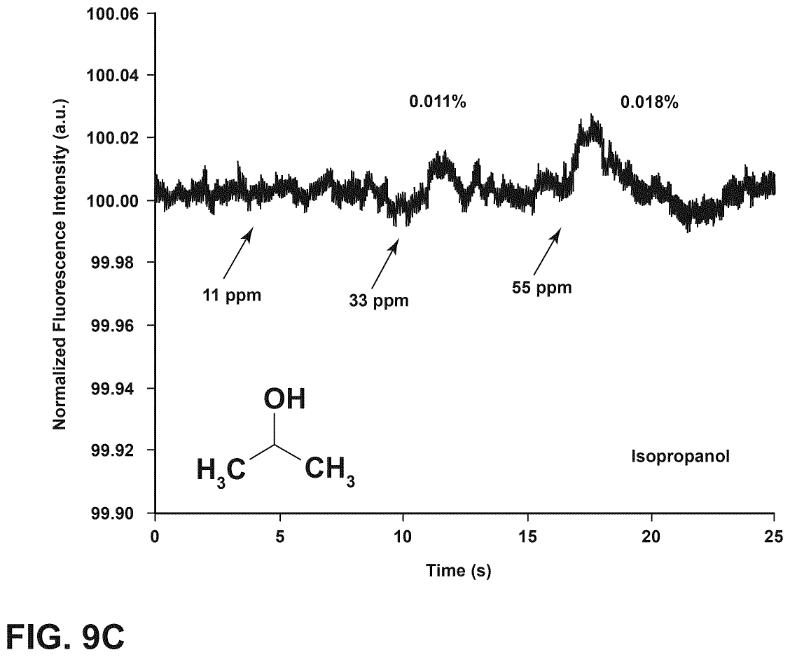

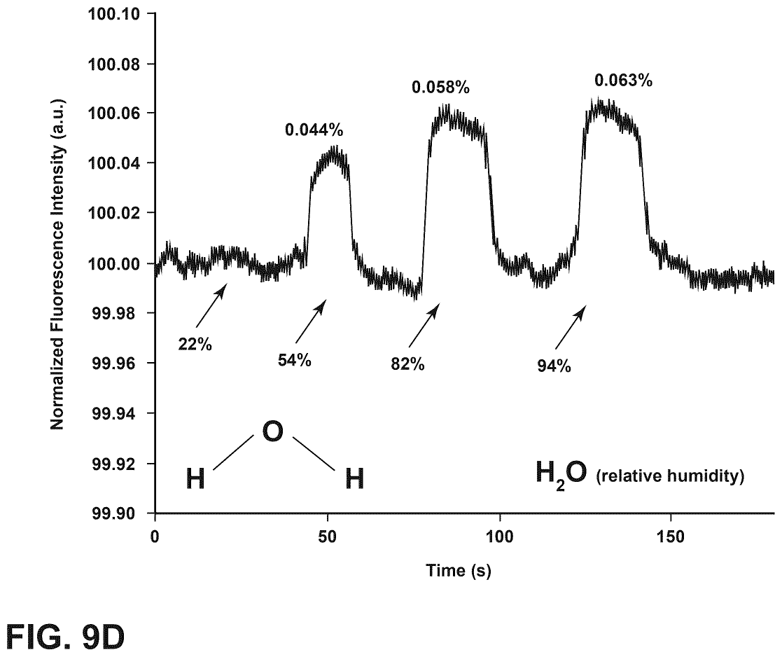

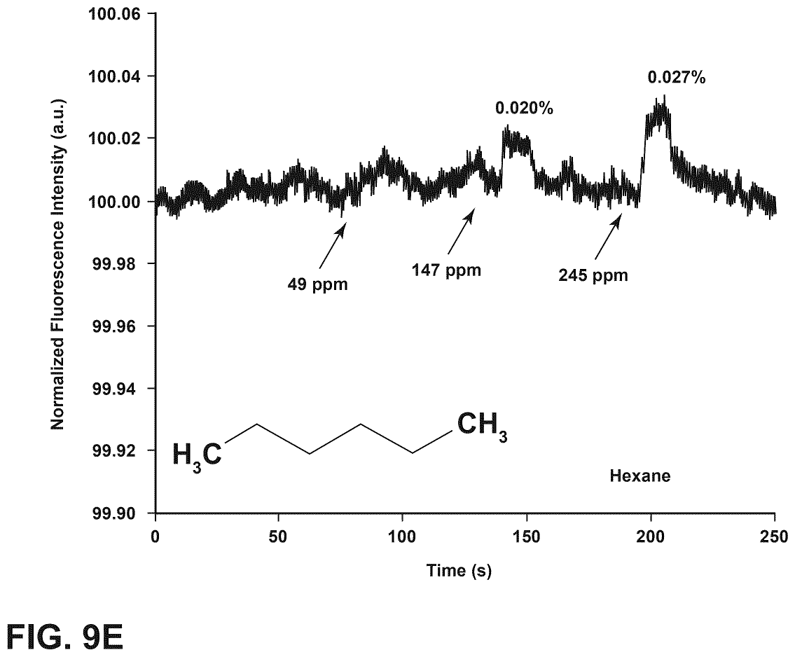

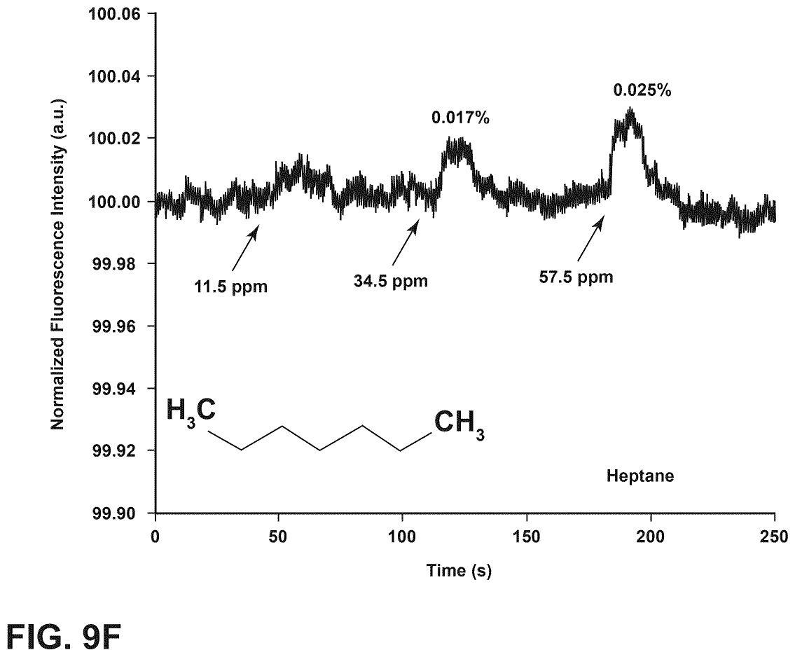

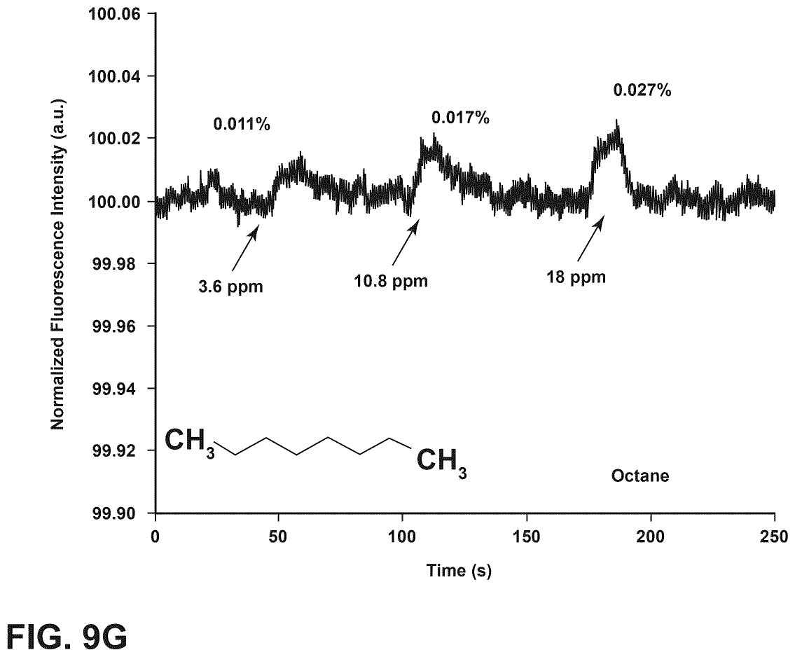

FIGS. 9A-G show the fluorescence responses to the common potential interferents: acetone, ethanol, isopropanol, water (relative humidity), hexane, heptane, and octane. All tested under the same conditions as performed for THC and CBD.

Trace level detection of THC and CBD on the substrate was also tested. The compounds TFT-C6 and TFT-C4 were self-assembled to form aggregates by injecting 0.1 ml of the chloroform solution of TFT-C6 and TFT-C4 (both 6 mg/mL) into a vial containing 1 mL of methanol and aging for 1 day at room temperature. The resulting assemblies that were suspended in solution that could be cast onto various substrates and into a quartz tube.

Optical chambers for fluorescent sensing were prepared by casting 10 μL of the aggregate suspension in methanol inside a quartz tube about 0.3 cm away from the air-inlet. The remaining methanol was removed from the quartz tube by a capillary. Then, the resulting aggregates inside the quartz tube were dried for 1 min by a blower. Fluorescence quenching experiments by analytes were performed on a prototype detection device, which used a 380 nm LED lamp as the excitation light source. After a diluted solution of analytes (e.g. THC) in methanol was deposited onto a polytetrafluoroethylene (PTFE) film and dried in air, the PTFE film was inserted into the detector where the sample was vaporized at 170° C. by a thermal desorber and pumped into the optical chamber containing the sensing materials (air pump rate, 200 ml min⁻¹). Notably, the temperature of the optical chamber was approximately 60° C. during the detection process.

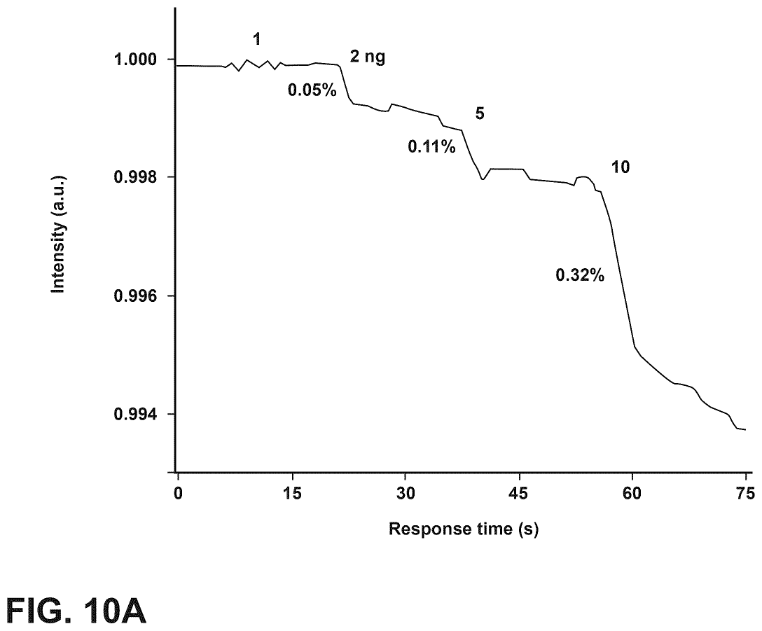

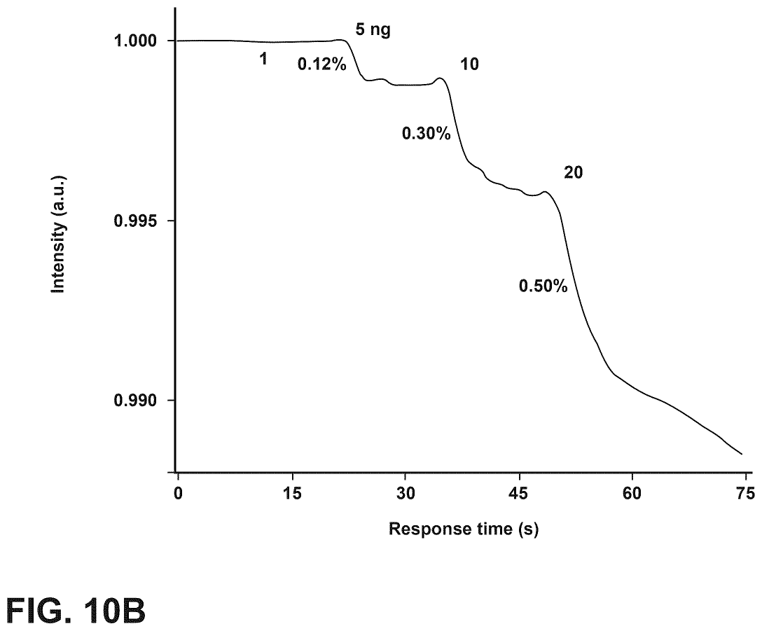

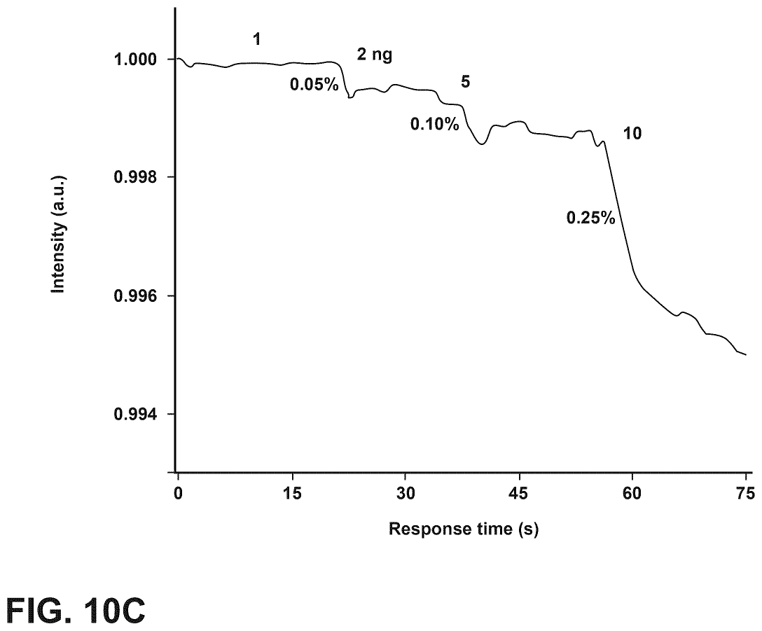

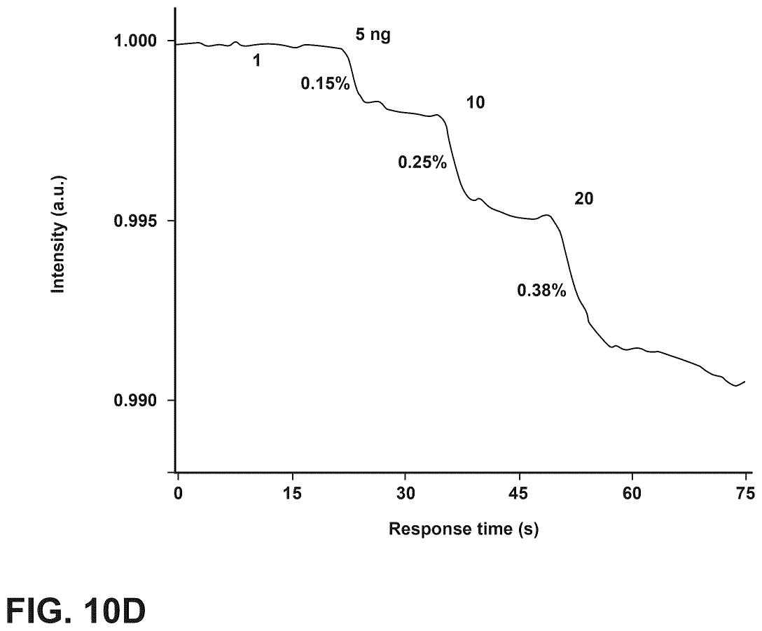

FIG. 10A shows the fluorescence responses of TFT-C6 thin films towards 1, 2, 5, and 10 ng of THC deposited on a substrate under 170° C. instant heating, which was believed to be sufficient to vaporize all the solid sample. FIG. 10B shows the results of repeating the same experiment with 5 ng, 10 ng, and 20 ng of THC.

FIG. 10C shows the fluorescence responses of TFT-C4 thin films towards 1, 2, 5, and 10 ng of THC deposited on a substrate under 170° C. instant heating, which was believed to be sufficient to vaporize all the solid sample. FIG. 10D shows the results of repeating the same experiment with 5 ng, 10 ng, and 20 ng of THC.

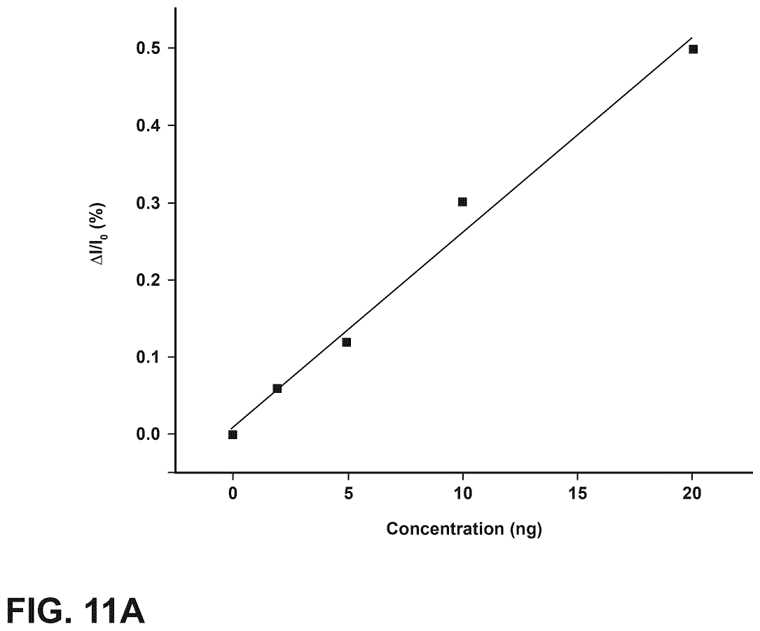

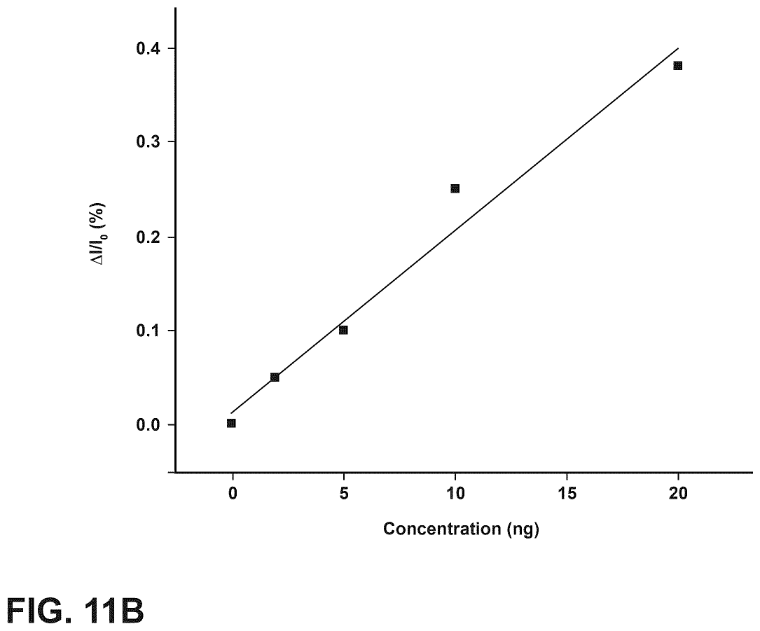

FIGS. 11A-B show the linear fitting curves for LOD calculation of fluorescence responses of TFT-C6 and TFT-C4, respectively, towards vaporized THC. The limit of detection (LOD) was determined by equation (iii):

$$LOD = 3 \times {RMS}_{Noise} / slope \qquad ( iii )$$

where RMS_{Noise} is calculated with equation (ii)

$${RMS}_{Noise} = \sqrt{\frac{\sum {( {I}_{i} - {I}_{c} )}^{2}}{N \times {( {I}_{0} )}^{2}}} \qquad ( ii )$$

The LOD of TFT-C6 and TFT-C4 was estimated as 0.36 ng and 0.38 ng, respectively. Another fluorescent sensor compound was also synthesized, having the structure shown:

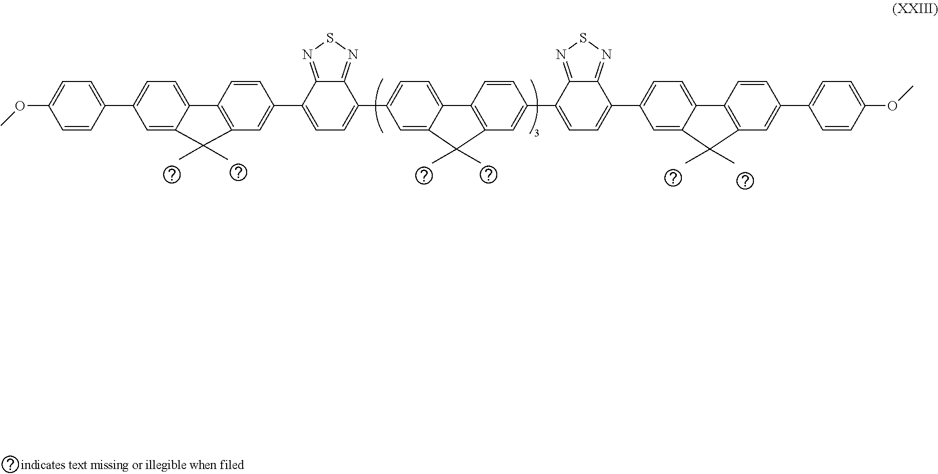

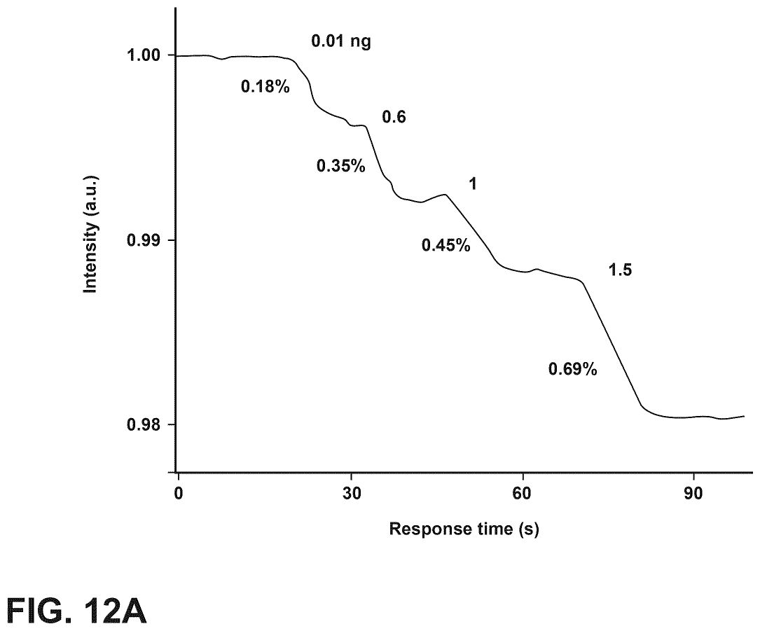

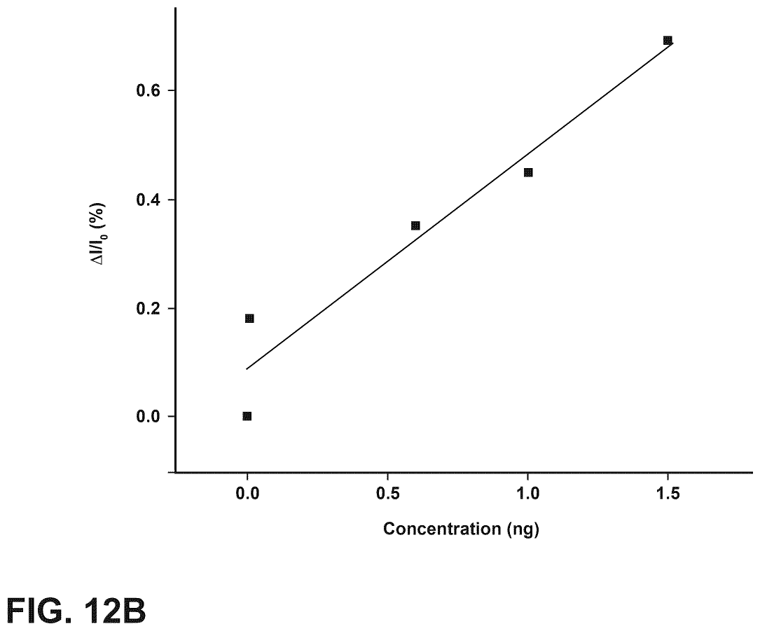

Compound XXIII was deposited in a quartz tube as described above. Compound XXIII was also tested by depositing THC in amounts of 0.01 ng, 0.6 ng, 1 ng, and 1.5 ng on a substrate and heating the THC at 170° C. FIG. 12A shows the fluorescence response. FIG. 12B shows the linear fitting curve for LOD calculation of the fluorescence responses using equations (ii) and (iii). The LOD of compound (XXIII) was estimated as 0.0013 ng, or 1.3 pg.

Testing of THC condensed with saliva shows the feasibility of detecting THC in real breath sample when moisture and many other compounds and bio-species present may interfere the sensor response. The sensor response was tested for 1 μl saliva deposited on a PTFE strip. Further, the sensor response was tested for 1 μL saliva co-deposited on the same PTFE strip with varying amounts of THC (10-100 pg). Saliva was used to mimic the biofluid condensed from exhaled breath, a simple way to test the potential interference on sensor performance. The sensor testing was performed with the same procedure using the same prototype detector equipped with front-end heater as described above. The sensor molecule used was also the same as used in Example 4, shown as Structure XXIII.

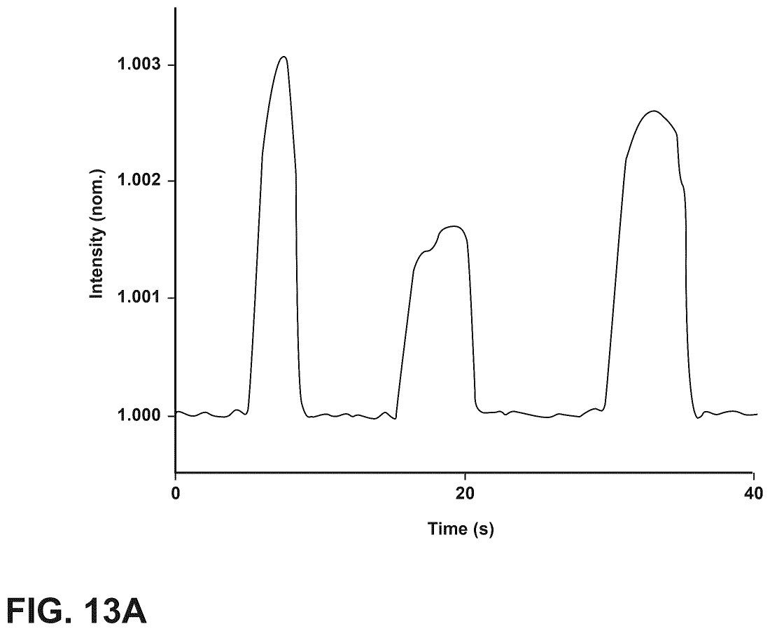

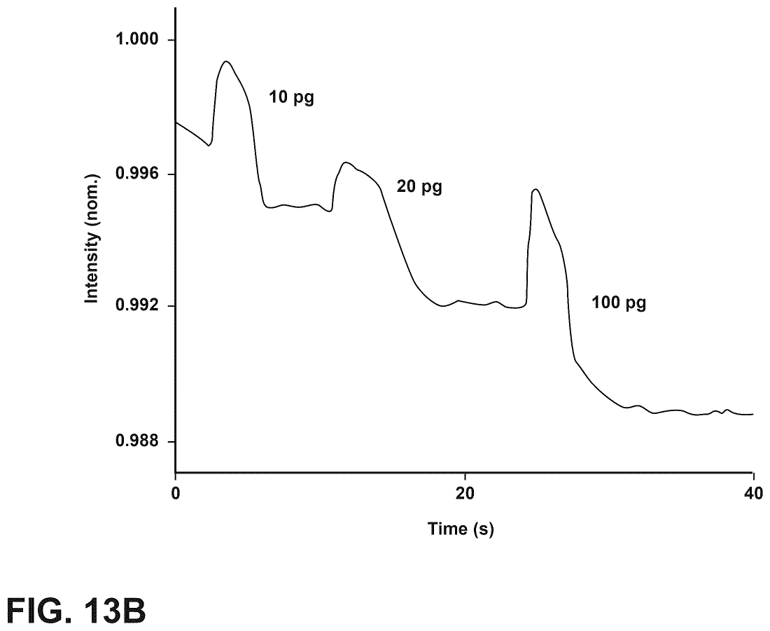

FIG. 13A shows sensor results using 1 μl saliva deposited on the PTFE testing strip, and measured with the prototype detector equipped with frontend heater using the sensor molecule XXIII. As can be seen, a quick reversible response was observed with a time span of ^{~}5 seconds. FIG. 13B shows sensor results using 1 μL saliva co-deposited with varying amount of THC (10, 20, 100 pg) on the PTFE testing strip, and measured with the prototype detector equipped with frontend heater using the molecule XXIII. The irreversible response (emission quenching) to THC can be clearly distinguished from the quick reversible response caused by the saliva (mostly due to the moisture). The response magnitude (relative decrease in emission intensity) is consistent with the results shown previously from the testing with pure THC sample. Thus, THC can be reliably detected when mixed with biofluids like saliva. Accordingly, detecting THC from a real breath sample condensed on a substrate can also be performed.

It will be understood by one having ordinary skill in the art that construction of the described disclosure and other components is not limited to any specific material. Other exemplary embodiments of the disclosure disclosed herein may be formed from a wide variety of materials, unless described otherwise herein.

According to one aspect of the present disclosure, a molecular sensor for detecting marijuana, includes a compound having a structure of Y-[D]ₙ-A-Z, D is an electron donor group according to Formula (I):

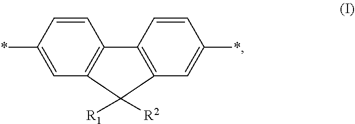

A is an electron acceptor group according to Formula (II):

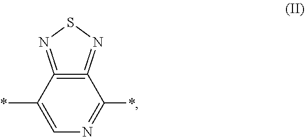

Y is R₃, R₃-A, or R₃-D-A, wherein Z is R₃ or [D]ₘ-R₃, n is an integer between 1 to 5, m is an integer between 1 to 3, R₁ and R₂ are each an alkyl chain, R₃ is an alkoxyphenyl end group.

According to another aspect of the present disclosure, R₁ and R₂ are the same alkyl chain.

According to yet another aspect of the present disclosure, R₁ and R₂ are alkyl chains having from 1 to 10 carbon atoms.

According to another aspect of the present disclosure, the compound is according to Formula (XXIV):

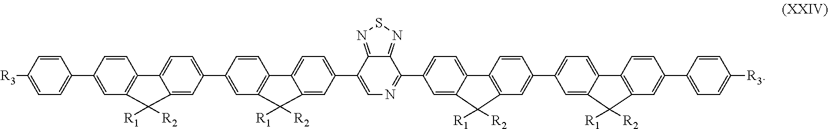

According to yet another aspect of the present disclosure, R₁ and R₂ are independently alkyl groups selected from the group consisting of —CH₃, —C₂H₅, —C₃H₇, —C₄H₉, —C₅H₁₁, C₆H₁₃, C₇H₁₅, and C₈H₁₇.

According to another aspect of the present disclosure, R₃ is selected from the group consisting of Formula (XI), Formula (XII), Formula (XIII), Formula (XIV), Formula (XV), Formula (XVI), Formula (XVII), Formula (XVIII), Formula (XIX), and Formula (XX):

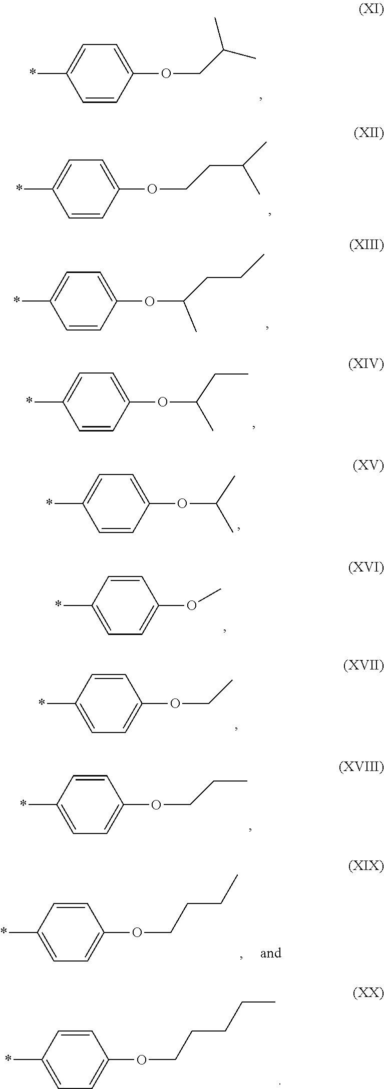

According to yet another aspect of the present disclosure, R₃ is Formula (XVI).

According to another aspect of the present disclosure, R₁ and R₂ are both —C₆H₁₃.

According to yet another aspect of the present disclosure microribbons, the microribbons comprise the compound having a structure of Y-[D]ₙ-A-Z.

According to another aspect of the present disclosure, a fluorescent sensor for detecting tetrahydrocannabinol (THC) or cannabidiol (CBD) includes, a porous film, the porous film includes an oligomer including fluorescent electron donor-acceptor molecules in a conjugation structure, a housing, an inlet and an outlet, the porous film is positioned between the inlet and the outlet, a light excitation source, and a fluorescence detector.

According to yet another aspect of the present disclosure, the oligomer comprises a general structure of Y-[D]ₙ-A-Z, wherein D is an electron donor group according to Formula (I):

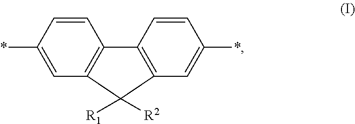

A is an electron acceptor group according to Formula (II):

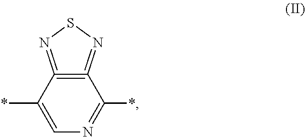

Y is R₃, R₃-A, or R₃-D-A, Z is R₃ or [D]ₘ-R₃, n is an integer between 1 to 5, wherein m is an integer between 1 to 3, wherein R₁ and R₂ are each an alkyl chain, R₃ is an alkoxyphenyl end group.

According to another aspect of the present disclosurem a condensing surface, the condensing surface is configured to collect a gaseous sample with a first concentration as a condensate, and a heater, the heater is configured to evaporate the condensate as a vapor with a second concentration, the second concentration is greater than the first concentration.

According to yet another aspect of the present disclosure, the gaseous sample is in the form of exhaled breath of a person.

According to another aspect of the present disclosure, the condensing surface is in the form of a membrane.

According to yet another aspect of the present disclosure, the oligomer will have a decrease fluorescent response in the presence of THC or CBD.

According to another aspect of the present disclosure, a method of detecting tetrahydrocannabinol (THC) or cannabidiol (CBD) in breath, including exposing a fluorescent sensor compound to a breath sample, the fluorescent sensor compound comprises an oligomer with a general structure of Y-[D]ₙ-A-Z, wherein D is an electron donor group according to Formula (I):

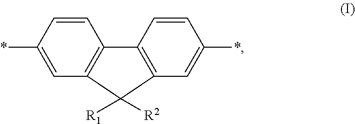

A is an electron acceptor group according to Formula (II):

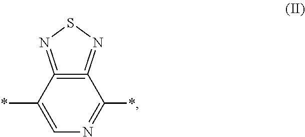

Y is R₃, R₃-A, or R₃-D-A, wherein Z is R₃ or [D]ₘ-R₃, n is an integer between 1 to 5, m is an integer between 1 to 3, R₁ and R₂ are each an alkyl chain, R₃ is an alkoxyphenyl end group, correlating a fluorescence response of the fluorescent sensor compound to a target threshold, and outputting an indicator corresponding to presence or absence of said THC or said CBD.

According to another aspect of the present disclosure, a method for detecting tetrahydrocannabinol (THC) or cannabidiol (CBD) in a gaseous sample, includes measuring a first fluorescence response of a porous film to establish a threshold, the porous film comprises an oligomer with a general structure of Y-[D]ₙ-A-Z, wherein D is an electron donor group according to Formula (I):

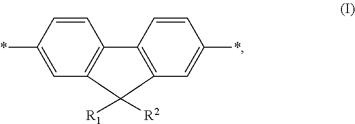

A is an electron acceptor group according to Formula (II):

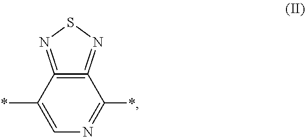

Y is R₃, R₃-A, or R₃-D-A, wherein Z is R₃ or [D]ₘ-R₃, where is an integer between 1 to 5, m is an integer between 1 to 3, R₁ and R₂ are each an alkyl chain, wherein R₃ is an alkoxyphenyl end group. The method further includes exposing said gaseous sample to a porous film, measuring a second fluorescence response of the porous film, the first fluorescence response is larger than the second fluorescence response when said gaseous sample comprises THC and/or CBD, and outputting a response if said gaseous sample contains THC and/or CBD.

According to yet another aspect of the present disclosure, the oligomer is of the formula (XXI) or (XXII):

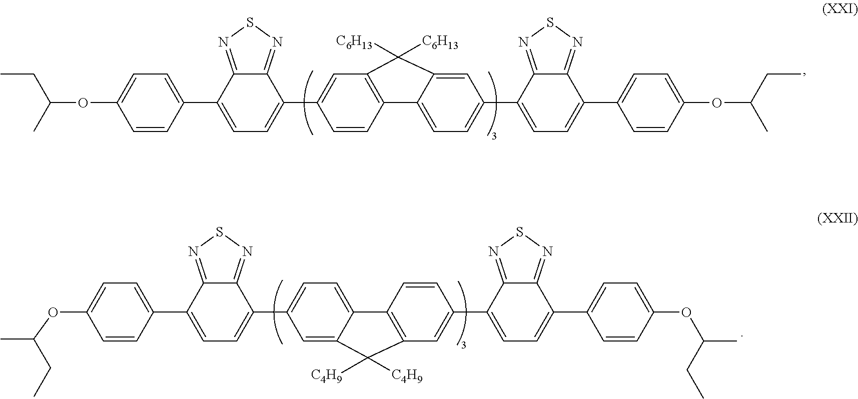

According to another aspect of the present disclosure, the method further includes condensing said gaseous sample onto the porous film to form a condensate, and heating the condensate to at least 60° C.

According to yet another aspect of the present disclosure, the gaseous sample contains one of alcohol, acetone, or relative humidity above 40%.

According to another aspect of the present disclosure, a limit of detection of THC and CBD is about 0.22 ppb and about 0.04 ppb, respectively.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A molecular sensor for detecting marijuana, comprising:
a compound having a structure of Y-[D]ₙ-A-Z, wherein D is an electron donor group according to Formula (I): 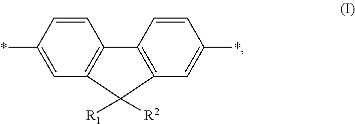
wherein A is an electron acceptor group according to Formula (II): 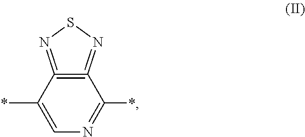
wherein Y is R₃, R₃-A, or R₃-D-A, wherein Z is R₃ or [D]ₘ-R₃, wherein n is an integer between 1 to 5, wherein m is an integer between 1 to 3, wherein R₁ and R₂ are each an alkyl chain, wherein R₃ is an alkoxyphenyl end group.

2. The molecular sensor of claim 1, wherein R₁ and R₂ are the same alkyl chain.

3. The molecular sensor of claim 2, wherein R₁ and R₂ are alkyl chains having from 1 to 10 carbon atoms.

4. The molecular sensor of claim 1, wherein the compound is according to Formula (XXIV):

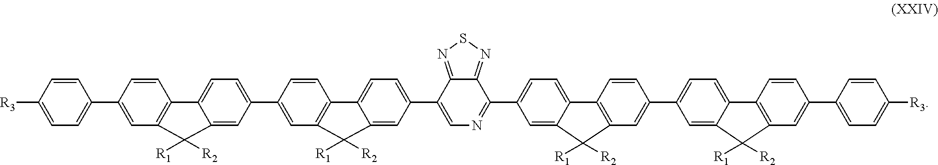

5. The molecular sensor of claim 1, wherein R₁ and R₂ are independently alkyl groups selected from a group consisting of —CH₃, —C₂H₅, —C₃H₇, —C₄H₉, —C₅H₁₁, C₆H₁₃, C₇H₁₅, and C₈H₁₇.

6. The molecular sensor of claim 1, wherein R₃ is selected from a group consisting of Formula (XI), Formula (XII), Formula (XIII), Formula (XIV), Formula (XV), Formula (XVI), Formula (XVII), Formula (XVIII), Formula (XIX), and Formula (XX):

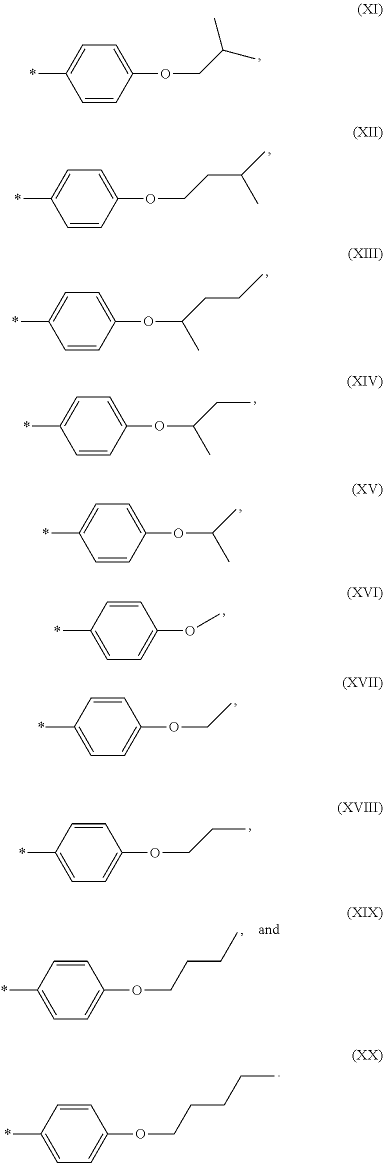

7. The molecular sensor of claim 6, wherein R₃ is Formula (XVI).

8. The molecular sensor of claim 1, wherein R₁ and R₂ are both —C₆H₁₃.

9. The molecular sensor of claim 1, further comprising:
microribbons, wherein the microribbons comprise the compound having the structure of Y-[D]ₙ-A-Z.

10. A fluorescent sensor for detecting tetrahydrocannabinol (THC) or cannabidiol (CBD) comprising:
a porous film, wherein the porous film comprises an oligomer including fluorescent electron donor-acceptor molecules in a conjugation structure;
a housing;
an inlet and an outlet, wherein the porous film is positioned between the inlet and the outlet;
a light excitation source; and
a fluorescence detector.

11. The fluorescent sensor for detecting tetrahydrocannabinol (THC) or cannabidiol (CBD) of claim 10, wherein the oligomer comprises a general structure of Y-[D]ₙ-A-Z, wherein D is an electron donor group according to Formula (I): 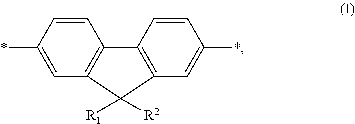
wherein A is an electron acceptor group according to Formula (II): 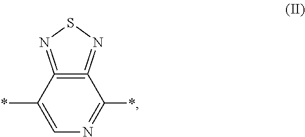
wherein Y is R₃, R₃-A, or R₃-D-A, wherein Z is R₃ or [D]ₘ-R₃, wherein n is an integer between 1 to 5, wherein m is an integer between 1 to 3, and wherein R₁ and R₂ are each an alkyl chain, wherein R₃ is an alkoxyphenyl end group.

12. The fluorescent sensor for detecting tetrahydrocannabinol (THC) or cannabidiol (CBD) of either claim 11, further comprising:
a condensing surface, wherein the condensing surface is configured to collect a gaseous sample with a first concentration of THC or CBD as a condensate; and
a heater, wherein the heater is configured to evaporate the condensate as a vapor with a second concentration of THC or CBD, wherein the second concentration is greater than the first concentration.

13. The fluorescent sensor for detecting tetrahydrocannabinol (THC) or cannabidiol (CBD) of claim 12, wherein the gaseous sample is taken from exhaled breath of a person.

14. The fluorescent sensor for detecting tetrahydrocannabinol (THC) or cannabidiol (CBD) of claim 12, wherein the condensing surface is in the form of a membrane.

15. The fluorescent sensor for detecting tetrahydrocannabinol (THC) or cannabidiol (CBD) of claim 10, wherein the oligomer will have a decreased fluorescent response in the presence of THC or CBD.

16. A method for detecting tetrahydrocannabinol (THC) or cannabidiol (CBD) in a gaseous sample, the method comprising:
measuring a first fluorescence response of a porous film to establish a threshold, wherein the porous film comprises an oligomer with a general structure of Y-[D]ₙ-A-Z, wherein D is an electron donor group according to Formula (I): 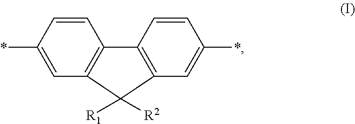
wherein A is an electron acceptor group according to Formula (II): 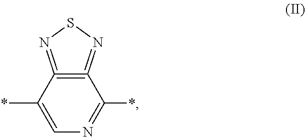
wherein Y is R₃, R₃-A, or R₃-D-A, wherein Z is R₃ or [D]ₘ-R₃, wherein n is an integer between 1 to 5, wherein m is an integer between 1 to 3, wherein R₁ and R₂ are each an alkyl chain, wherein R₃ is an alkoxyphenyl end group;
exposing said gaseous sample to the porous film;
measuring a second fluorescence response of the porous film, wherein the first fluorescence response is larger than the second fluorescence response when said gaseous sample comprises THC or CBD; and
outputting a response if said gaseous sample contains THC or CBD.

17. The method of detecting tetrahydrocannabinol (THC) or cannabidiol (CBD) in a gaseous sample of claim 16, wherein the oligomer is of the formula (XXI) or (XXII):

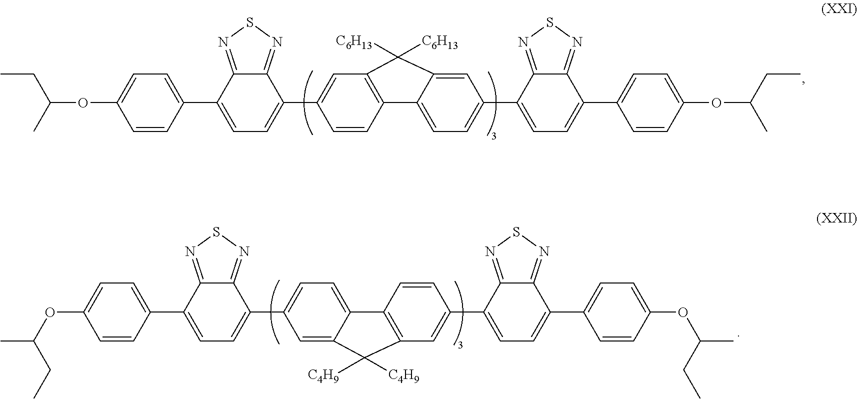

18. The method of detecting tetrahydrocannabinol (THC) or cannabidiol (CBD) in a gaseous sample of claim 16, wherein the method further comprises:
condensing said gaseous sample onto the porous film to form a condensate; and
heating the condensate to at least 60° C.

19. The method of detecting tetrahydrocannabinol (THC) or cannabidiol (CBD) in a gaseous sample of claim 16, wherein said gaseous sample contains one of an alcohol, acetone, or relative humidity above 40%.

20. The method of detecting tetrahydrocannabinol (THC) or cannabidiol (CBD) in a gaseous sample of claim 16, wherein a limit of detection of THC and CBD is about 0.22 ppb and about 0.04 ppb, respectively.
